(19) United States
(12) Patent Application Publication
(10) Pub. No.: US 2026/0158223 A1
(43) Pub. Date: Jun. 11, 2026

(54) METHOD AND APPARATUS TO REDUCE THE CUBIC VOLUME IN CARTRIDGES AND CARPULES

(71) Applicant: Thomas Kuracina, Carson City, NV (US)

(72) Inventor: Thomas Kuracina, Carson City, NV (US)

(21) Appl. No.: 19/179,920

(22) Filed: Apr. 15, 2025

Related U.S. Application Data
(60) Provisional application No. 63/728,193, filed on Dec. 5, 2024.

(51) Int. Cl. A61M 5/315; A61M 5/24; A61M 5/31

(52) U.S. Cl. CPC A61M 5/31511; A61M 5/2422; A61M 5/2459; A61M 2005/3123; A61M 2005/31518

(57) ABSTRACT

Method and apparatus to reduce the volume in medical cartridges and carpules with the use of volume displacing plungers that occupy space within one or more interior spaces of the cartridges and carpules. The volume displacing plungers are also equipped with one or more interior cavities or flow passages that facilitate a flow of a fluid through the cartridges and carpules. Some volume displacing plungers are also adapted to cooperate with features of the cartridges and carpules that house them to form air-tight and liquid-tight seals that at least partially determine a fluid pathway through the cartridges and carpules.

### CROSS-REFERENCE TO A RELATED APPLICATION

This application is related to and claims the benefit of filing date of the following U.S. Provisional Patent Application: Ser. No.: 63/728,193, entitled: LOW DEADSPACE CARTRIDGES AND CARPULES, filed Dec. 5, 2024. The above-referenced provisional patent application is incorporated herein by reference in its entirety.

### FIELD

The present invention relates to reduced-volume cartridges and carpules, for delivering fluids from dispensing devices, that may include syringes, pen injectors, auto-injectors and pumps, configured with a cartridge or carpule with a fillable hollow barrel with a moveable plunger, piston or stopper disposed in the hollow barrel through an open proximal end with a first inside diameter, and an opposing funneled hollow distal end configured with a funneled interior cavity with reduced inside diameter with an open distal end covered by a pierceable membrane, septum or diaphragm. Cartridges and carpules are glass-or polymer-based hollow cylindrical containers sold empty or filled with a medical fluid, that may conform with the design, dimensions, materials, performance and test methods for cylinders and plungers recommended by the International Organization for Standardization (ISO). ISO 13926-1: 2018 specifies the parameters used to produce and test pen-injectors for medical use. ISO 13926-2:2018 specifies the parameters used to produce rubber plunger stoppers for medical use. ISO 13926-3:2018 specifies the parameters used for seals of rubber plunger stoppers for medical use. ISO 21881:2019 specifies the parameters used to produce and test sterile packaged ready for filling glass cartridges. The reduced-volume cartridges and carpules of the present invention may comprise the design, dimensions, materials, performance and test methods conforming to the specifications of ISO 21881:101A9 and ISO 13926-1:101A 8. The terms cartridges, carpules and vials are interchangeably used throughout this specification. The terms medicament, fluid, liquid, and gas are interchangeably used to describe an injectable medical agent throughout this specification. The terms membrane, septum, diaphragm, and lined rubber membrane are interchangeably used to describe a pierceable material throughout this specification.

### BACKGROUND

Medical devices, such as syringes and auto-injectors are well known in the art for delivering medical fluids, including but not limited to, medication, therapeutic and prophylactic agents, analgesics, narcotic analgesics, vaccines, biologics, biosimilars, peptide hormones, GLP-1 receptor agonists, amycretin, SGLT2i inhibitors, PD-1 inhibitors, anesthetics, human hyaluronidases, gases and the like. Injection pens are sold either empty or with prefilled cartridges and are provided in a single dose or multi-dose configuration. The cartridge is provided with a moveable proximal elastomeric plunger and and elastomeric membrane or diaphragm designed as barriers to maintain the sterility of a prefilled medical fluid in the hollow barrel whether a single dose or multiple injections are dispensed. The plunger is held in place in the hollow barrel by a static friction that must be overcome before the plunger will start to move. A break-loose force is required to move the plunger in the hollow barrel. The distal end of an injection pen is configured with a cartridge holder adapted to receive a cartridge. A glide force is the force required to overcome the dynamic friction of the plunger moving through the barrel of the cartridge or syringe and the hydraulic resistance of the fluid being pushed through the small needle lumen. The injection pen is configured with an exterior wall adapted to receive the open end of a cap. The injection pen is configured with a cartridge holder, with an interior adapted to receive a cartridge, and a distal end adapted to receive a separate needle hub with a needle. A new needle is recommended for each injection.

In the 2012 Accidental Intravenous Infusion of Air study published in the Infusion Nurses Society, Wilkins and Unverdorben observe that a spherical bubble as small as 0.2 mm may cause detectable cerebral ischemia. Any air must be removed, or segregated from the fluid in a syringe, prefilled syringe, or injector pen or insulin pump prior to dispensing the dose. Air bubbles are known to naturally form in the cartridge of an insulin pen when the pen warms after it is removed from a refrigerator. Priming the insulin pen before each injection to remove any air bubbles that may have accumulated in the needle and cartridge also ensures an accurate dose of insulin. Air bubbles may also form in the insulin cartridge as it warms after the cartridge is removed from a refrigerator before it is placed into an injection pen or pump. A small amount of fluid must be pushed from the hollow barrel, needle hub (in a Luer syringe), and needle lumen to remove the air from the fluid before each dose can be dispensed. This priming loss is known as wastage, and any fluid retained within a syringe, injection pen or pump after each dose has been dispensed is known as dead-volume or deadspace wastage. If less than a 0.50 ml dose remains in the hollow barrel of the cartridge of an injection pen designed to deliver a series of full 0.5 ml doses, the injection pen locks and will not dispense any fluid. Approximately 0.15 ml (or 30% of a 0.5 ml dose) remains in the interior “funnel” cavity 50 of the standard cartridge 1 after the dose(s) are dispensed. Early testing of the reduced-volume cartridge of the present invention reduces the wastage, from approximately 0.15 ml in the interior deadspace cavity 50 of the standard cartridge 1, to (best case) approximately 0.03 ml (or 6% of a 0.5 ml dose), achieving an approximate 80% reduction of wastage compared with a standard cartridge. A 0.10 ml savings from each reduced-volume cartridge of the present invention equals 100,000 ml of medical fluid for every 1 million cartridges produced, equaling approximately 200,000 new 0.5 ml doses.

The World Health Organization has estimated the number of people living with diabetes rose from 200 million in 1990 to 830 million in 2022. Even though diabetes prevalence has increased rapidly in low-and middle-income countries, more than half of the population living with diabetes in those countries do not take medication for their diabetes. In 2021, the Centers for Disease Control and Prevention (CDC) estimated 38.4 million adults in the United States (11.6% of the population) had diabetes. A recent study in the Lancet estimates more than 1 billion people were living with obesity in 2022, doubling among adults since 1990. Obesity is known to cause cardiovascular disease, diabetes and cancer. The obesity drug market is expected to grow at a CAGR of 32.3% and exceed $173.5 billion by 2031. In addition to insulin, the development of GLP-1 agonists, sub-Q PD-1 inhibitors, mono-clonal antibodies, biologics, and biosimilars continue to increase the demand for injection pens. A recent market report has estimated the injectable drug-delivery market was valued at $483.5 billion in 2019, growing at an estimated CAGR of 13.1% through 2032 and valuing the market at $2.65 trillion. With tens of billions of injections being dispensed globally every year, any meaningful reduction in drug wastage has significant implications to improve supply chain resiliency. The reduced wastage from a reduced-volume injection apparatus can be combined into additional doses for patients, helping to mitigate drug shortage issues, as well as creating a measurable revenue-producing and reduced cost-per-dose benefit for manufacturers.

A number of patents and published applications describe medical dispensing devices that include a means to reduce the volume within a cartridge or syringe. These are U.S. Pat. No. 2,887,108 (Kendall), U.S. Pat. No. 3,331,538 (Higgins), U.S. Pat. No. 3,672,369 (Brown), U.S. Pat. No. 5,240,322 (Haber et al.), U.S. Pat. No. 5,240,323 (Haber et al.), U.S. Pat. No. 5,658,259 (Pearson et al.), U.S. Pat. No. 5,713,866, (Wilmot), U.S. Patent. No. 6,210,369 (Wilmot et al.), EP3897787B1 (Mariani), and U.S. Pat. No. 10,603,440 (McCaffrey et al.).

In EP3897787B1, Mariani teaches an injection kit 200 with a multi-piece piston 1 comprising an axial end 20 with at least one through opening 26, and preferably two as illustrated in the figures, which extend from the outer surface 4A to the inner surface 4B of the axial extension 4. The traverse through opening 26 includes at least one proximal wall with an opening in communication with continuous, concentric interior “cavity 6 (and all the portions thereof) hence exhibits a diameter that decreases progressively from, located in a point corresponding to the skirt 2, to a second axial end 20” ¶0024 . The second (distal) axial end 20 includes a single opening 26 in communication with a (through) cavity 6, as shown in FIG. 2, defined by an inner surface 4A, configured between a third stretch 11 and a fourth stretch 12, as shown in FIG. 4, “which has a smaller diameter than the third stretch 11, thus defining a shoulder 14 between them” ¶0022. The proximal end of the needle 212 is configured with a jacket 215 and a stud 220, as shown in FIGS. 10, 10A and 11, with a width exceeding the inside diameter of the (through) cavity 6 configured between stretch 11 and stretch 12, as shown in FIG. 4. When the multi-piece piston 1 is moved to the distal end of the tubular vial 100, the proximal end of the needle 212, jacket 215 and stud 220 advance through the (through) cavity 6 configured between the fourth stretch 12 (illustrated without cross-hatching in FIG. 4) and the third stretch 11, allowing the stud 220 to engage the shoulder 14 configured inside stretch 11, as the selectively engaged needle 212 and piston are moved away from the bushing member 214. The engagement between the stud 220 with the shoulder 14 of the multi-piece piston 1 allow the user to selectively retract the needle 212 into the vial 100 in FIG. 9 ¶0008. The multi-piece piston comprises a first portion 6D with first axial end 18 configured with a core portion C that extends to a second portion 6P unitarily formed of a rigid plastic with an axial extension 4 with distal axial end. An elastomeric skirt 2 is insert-molded onto the core portion C due to the design of the axial extension 4 configured to decrease progressively from the skirt 2 to the axial end 20. The multi-piece piston 1, comprises a continuous interior cavity 6, configured with the core portion C, stretch 12, stretch 11 and stretch 10 with inside diameters successively increasing the from the inside diameter of the opening 26 in stretch 12 of the axial end 20, as shown in FIG. 2, to the skirt 2, making the piston 1 and skirt 2 impossible to manufacture as a single piece. The interior cavity of stretch 11 is far wider than the narrow opening in cavity 6 in stretch 12 (see FIG. 4) with a width “in the range between 0.3 mm and 0.7 mm.” A solid molding core, with an outside diameter capable of injection molding the interior cavity of stretch 11 of a one-piece piston 1, would be unable to be extracted through the small opening of the cavity 6 between stretch 11 and stretch 12 without completely destroying the axial end 20 of the narrower stretch 12. In U.S. Pat. No. 2,887,108, Kendall teaches a piston 18 with an extension 27′ including axial grooves 28 intersecting with grooves 29 in the outer face of the extension. In U.S. Pat. No. 5,713,866, Wilmot teaches a plunger 28 with the forward portion 104 (nipple) with a central bore 120 extending into (only) the forwardmost surface 110 so that the nipple can receive the rearward end of the needle 34 when the nipple reaches its forwardmost point of travel, and least one axially extending groove 116 disposed in an outer peripheral surface of the plunger 28. The plunger 28, comprises a central bore 120 in the forwardmost surface 110 and includes an open end and a closed end and closed sides configured to trap any air that may reside with any fluid within in the space 106 (interior cavity) of the cartridge 12 during use.

### SUMMARY

The present inventions generally relate to a cartridge or carpule comprising a hollow barrel, for accommodating fluid, configured with a hollow proximal portion with a first inside diameter with an open end, and a distal portion having a distal end with a reduced inside diameter with an interior cavity with a distal open end. A pierceable membrane covers the distal open distal end and may be secured by a cap over the distal open end. The volume-displacing plunger includes a body, with a distal end configured to reduce the space within the interior cavity of the hollow barrel. The distal end, of the body, includes an interior cavity or through passage configured between an open distal end and an open side wall, and configured to displace the space within the interior cavity of the hollow barrel.

One implementation of the present invention comprises a volume-displacing plunger configured to reduce a portion of the space for accommodating a liquid or gas within the hollow barrel of a cartridge or carpule before, during or after the fluid has been dispensed. One implementation of the present invention comprises a volume-displacing plunger configured with at least one interior cavity or through passage with at least one opening configured in at least a portion of a body of the volume-displacing plunger. Another implementation of the present invention comprises a volume-displacing plunger configured with at least one interior cavity configured between a plurality of openings in the body of the volume-displacing plunger.

One implementation of the present invention comprises a volume-displacing plunger with a body configured to reduce the space within the hollow barrel of a cartridge or carpule. One implementation of the present invention comprises a volume-displacing plunger with a distal end configured to reduce the space within the hollow barrel of a cartridge or carpule. One implementation of the present invention may a include a volume-displacing plunger with a conical portion and an elongated portion forming a funneled distal end configured to reduce the space within the hollow barrel of a cartridge or carpule. One implementation of the present invention may also include a volume-displacing plunger, configured to reduce the amount of medicine or fluid within the hollow barrel of a cartridge or carpule, with a body with an interior cavity or through passage, defined by at least one interior wall, having an open distal end in communication with a side port. Another implementation of the present invention may also include a volume-displacing plunger, configured to reduce the amount of medicine or fluid within the interior cavity of a cartridge or carpule, with a body having an interior cavity or through passage, defined by an interior wall, configured in a “T” or “Y” configuration, having a first distal opening, at least a second opening and third opening, configured in at least a portion of an exterior side wall.

Another implementation of the present invention may include a volume-displacing plunger, configured to reduce the space within the interior cavity of a cartridge or carpule, with a body with an interior cavity or through passage, configured by at least one opening configured within at least a portion of a distal facing end wall, and at least a portion of a first side wall, and at least a portion of a second sidewall. One implementation of the present invention may include a volume-displacing plunger, configured to reduce the space within the interior cavity of a cartridge or carpule, with an interior cavity or through passage, configured with at least one opening configured in a distal facing end wall, and at least one opening configured in a conical side wall. Another implementation of the present invention may include a volume-displacing plunger, configured to reduce the space within the interior cavity of a cartridge or carpule, with an interior cavity or through passage, configured with at least one opening in at least one portion of a distal end wall, intersecting with a second interior cavity, configured with at least one opening in at least a portion of a side wall, that may include at least a portion of a conical wall. One implementation of the present invention may also include a volume-displacing plunger, configured to reduce the amount of medicine or fluid within the interior cavity of a cartridge or carpule, with a body with an interior cavity or through passage defined by an interior wall, having a first opening intersecting with a second opening. One implementation of the present invention may also include a volume-displacing plunger, configured to reduce the amount of medicine or fluid within the interior cavity of a cartridge or carpule, with an interior cavity or through passage configured with a continuous opening, configured between a first opening, configured in at least a portion of a distal end wall, intersecting with at least a second opening, configured in a side wall, that may include at least a portion of a conical wall. One implementation of the present invention may also include a volume-displacing plunger, configured to reduce the amount of medicine or fluid within the interior cavity of a cartridge or carpule, with an interior cavity or through passage configured with a continuous opening configured between at least a portion of a distal facing end wall, and at least a portion of a conical side wall.

One implementation of the present invention may a include a cartridge or carpule, fillable, by inserting a hollow tube or needle, of a reservoir filled with fluid, through a membrane covering the distal open end of the cartridge, and moving the fluid from the reservoir into the hollow barrel of the cartridge. Another implementation of the present invention may a include a cartridge or carpule fillable through the distal opening at the reduced distal end, after a volume-displace plunger is disposed in a portion of the hollow barrel of the cartridge. Another implementation of the present invention may a include a cartridge or carpule fillable through the proximal opening in the proximal portion, after the distal opening in the reduced distal end is covered by a membrane that may include a cap positioned and crimped over the distal open end. Another implementation of the present invention may a include a cartridge or carpule, fillable through the membrane at the distal end, after a volume-displace plunger is positioned within the interior cavity of the cartridge or carpule.

These and other objects, features and advantages of the present invention will become evident in view of the following detailed description taken in conjunction with the accompanying drawings.

### BRIEF DESCRIPTION OF THE DRAWINGS

FIG. 1A is a cross-sectional side view of an example of a prior art cartridge or carpule with a moveable plunger disposed in a hollow barrel.

FIG. 1B is a cross-sectional side view of the prior art cartridge of FIG. 1A oriented in a horizontal position with a screw cap covering the distal end of the cartridge with a needle piercing a lined rubber membrane and air trapped in a corner of the hollow barrel.

FIG. 1C is a cross-sectional side view of the prior art cartridge of FIG. 1A disposed in a cartridge holder of a fluid dispensing apparatus with a plunger disposed in the hollow barrel of the cartridge and fluid remaining in an interior cavity of the funneled hollow distal end of the barrel.

FIG. 1D is a cross-sectional side view of a prior art needle hub with a needle.

FIG. 1E is a cross-sectional side view of the prior art cartridge of FIG. 1A in a vertical position with a needle piercing a membrane and air trapped in the hollow barrel.

FIG. 1F is a partial cut-away and cross-sectional side view of a prior art medical fluid dispensing device with a cartridge holder holding a cartridge filled with a medical fluid and a cap covering the cartridge holder.

FIG. 2A is a cross-sectional side view of one implementation of the reduced-volume cartridge of the present invention with an elastomeric volume-displacing plunger disposed in the hollow barrel for accommodating a liquid or gas.

FIG. 2B is a cross-sectional side view of one implementation of a fluid dispensing apparatus of the present invention with a cartridge holder holding the reduced-volume cartridge of FIG. 2A, configured with a funneled end of the volume-displacing plunger disposed in the funneled hollow distal end of the cartridge and a needle hub with a needle disposed in the interior cavity of the hollow distal end.

FIG. 2C is a cross-sectional side view of one implementation of the cartridge/needle apparatus 101B of the present invention in a vertical position with the funneled end of the volume-displacing plunger disposed in the funneled hollow distal end of the cartridge and air trapped in a portion of the interior cavity of the funneled hollow distal end at a safe distance from the needle.

FIG. 2D is a cross-sectional side view of one implementation of the cartridge/needle apparatus 101C of the present invention with a volume-displacing plunger with a funneled distal end with an interior cavity configured with an angled proximal interior wall configured to channel any air within the interior cavity to rise away from the needle.

FIG. 2E is a cross-sectional side view of one implementation of the cartridge/needle apparatus of FIG. 2D with a volume-displacing plunger configured with a distal end wall engaging a pierceable septum or membrane of rubber stopper.

FIG. 2F is a cross-sectional side view of one implementation of the cartridge/needle apparatus 101D of the present invention with a volume-displacing plunger with a funneled distal end with an interior cavity defined by a plurality of openings configured in the distal end.

FIG. 2G is a cross-sectional side view of the cartridge/needle apparatus of FIG. 2C, in axis 2G-2G, with distal end of the volume-displacing plunger displacing the majority of fluid in the interior cavity of the funneled hollow distal end of the reduced-volume cartridge.

FIG. 2H is a cross-sectional front view of the cartridge/needle apparatus of FIG. 2Dm in axis 2H-2H, with the distal end of the volume-displacing plunger displacing the majority of fluid in the funneled interior cavity of the funneled hollow distal end of the reduced-volume cartridge.

FIG. 2I is a cross-sectional front view of the reduced-volume cartridge of FIG. 2E in axis 2I-2I with distal end wall of the funneled distal end of the volume-displacing plunger engaging the proximal face of the membrane.

FIG. 2J is a cross-sectional front view of the cartridge/needle apparatus of FIG. 2E in axis 2J-2J with the distal end of the volume-displacing plunger displacing the majority of fluid in the conical portion of the funneled interior cavity of the funneled hollow distal end of the reduced-volume cartridge

FIG. 2K is a cross-sectional front view of the cartridge/needle apparatus of FIG. 2F in axis 2K-2K with a distal end of the volume-displacing plunger configured with a plurality of openings in the funneled distal end.

FIG. 2L is a cross-sectional side view of one implementation of the volume-displacing plunger of FIG. 2A configured with a body with the funneled distal end having an outside diameter <D2.

FIG. 2M is a cross-sectional side view of one implementation of the medical fluid delivery device of the present invention with a cartridge holder holding a reduced-volume cartridge with a volume-displacing plunger disposed in the hollow barrel and a removable cap attached to the medical fluid delivery device

FIG. 2N is a cross-sectional side view of one implementation of the volume-displacing plunger of the present invention configured with funneled distal end with an elongate opening in an exterior wall.

FIG. 2O is a full top view of the elastomeric volume-displacing plunger of FIG. 2A, configured with funneled distal end with a distal interior cavity with a solid proximal wall and an opposing opening in a distal facing end wall.

FIG. 2P is a full front view of the volume-displacing plunger of FIG. 2O, configured with distal end with a distal interior cavity with at least one axial interior radial side wall and an opposing opening in an exterior side wall.

FIG. 2Q is a full top view of the elastomeric volume-displacing plunger FIGS. 2D and 2E, configured with funneled distal end with a distal interior cavity with an angled solid proximal wall and an opposing opening in a distal facing end wall.

FIG. 2R is a full front view of the volume-displacing plunger of FIG. 2Q, configured with funneled distal end with a distal interior cavity with at least one axial interior side wall and an opposing opening in an exterior side wall.

FIG. 2S is a full top view of the elastomeric volume-displacing plunger of FIG. 2F, comprising a distal end configured with a distal interior cavity with a first axial interior side wall and an opposing second axial interior side wall, and a non-planer solid proximal wall and an opposing opening in a distal facing end wall.

FIG. 2T is a full front view of the volume-displacing plunger of FIG. 2S configured with funneled distal end with a distal interior cavity traversing across the outside diameter of the distal end.

FIG. 3A is a cross-sectional side view of one implementation of the reduced-volume cartridge of the present invention with a unitarily-formed elastomeric volume-displacing plunger comprising a funneled distal end, with a through passage, disposed in the hollow barrel of the cartridge.

FIG. 3B is a cross-sectional side view of the reduced-volume cartridge of the present invention with a unitarily-formed volume-displacing plunger with a funneled distal end disposed in the funneled interior cavity of the funneled hollow distal end of the reduced-volume cartridge.

FIG. 3C is a cross-sectional side view of the unitarily-formed volume-displacing plunger of FIG. 3A, configured with a funneled distal end with an interior through passage with a first opening in a distal end wall and a second opening configured in an exterior side wall.

FIG. 3D is a cross-sectional side view of the one-piece volume-displacing plunger of FIG. 3C configured with a funneled distal end with a through passage with an opening in the distal end wall.

FIG. 3E is a cross-sectional side view of one implementation of an injection molding tool comprising a method to mold the elastomeric volume-displacing plunger of the present invention in a single piece.

FIG. 4A is a cross-sectional side view of one implementation of the reduced-volume cartridge of the present invention with a volume-displacing plunger with features adapted to cooperate with the features of the reduced volume cartridge to form air-tight and liquid-tight seals that at least partially determine a fluid pathway through the cartridge and the volume-displacing plunger.

FIG. 4B is a full top view of the volume-displacing plunger of FIG. 4A, configured with a funneled distal end with an elongated distal interior cavity with a solid proximal wall and an opposing opening in a distal facing end wall.

FIG. 4C is a full front view of the volume-displacing plunger of FIG. 4B, configured with a funneled distal end with an elongate distal interior cavity with at least one axial interior radial wall and an opposing opening in an exterior side wall.

FIG. 4D is a cross-sectional side view of one implementation of the fluid dispensing apparatus of the present invention, configured with a volume-displacing plunger of FIG. 4A disposed within the hollow barrel of the reduced-volume cartridge.

FIG. 4E is a cross-sectional side view of the medical fluid dispensing apparatus of FIG. 4B, with the volume-displacing plunger positioned within the funneled interior cavity of the funneled hollow distal end of the reduced-volume cartridge.

FIG. 4F is a cross-sectional front view of the dispensing apparatus of FIG. 4D in axis 4F-4F, with fluid disposed within the conical portion of the funneled interior cavity of the funneled hollow distal end of the reduced-volume cartridge.

FIG. 4G is a cross-sectional front view of the dispensing apparatus of FIG. 4D in axis 4G-4G, with fluid disposed within the narrow tube portion of the funneled interior cavity of the funneled hollow distal end of the reduced-volume cartridge.

FIG. 4H is a cross-sectional front view of dispensing apparatus, of FIG. 4E, in axis 4H-4H, configured with the funneled distal end of the volume displacing plunger displacing the fluid previously disposed in the conical portion of the funneled interior cavity of the funneled hollow distal end of the reduced-volume cartridge.

FIG. 4I is a cross-sectional front view of the dispensing apparatus of FIG. 4E in axis 4I-4I, configured with the funneled distal end of the volume displacing plunger displacing the majority of fluid previously disposed in the narrow tube portion of the funneled interior cavity of the funneled hollow distal end of the reduced-volume cartridge.

FIG. 4J is an isometric view of the volume-displacing plunger of, FIG. 4A, with an interior cavity configured in the distal end with a first opening in the distal facing end wall, and a second opening in the exterior side wall.

FIG. 4K is a cross-sectional side view of one implementation of the medical fluid delivery device of the present invention with a cartridge holder holding a reduced-volume cartridge with a volume-displacing plunger disposed in the hollow barrel and a cap attached to the medical fluid delivery device.

FIG. 4L is a cross-sectional side view of the volume-displacing plunger, of FIG. 4A, comprising a proximal portion, and a funneled distal end with a conical portion and an elongate axial portion configured with an outside diameter ≥D2.

### DETAILED DESCRIPTION OF THE DRAWINGS

A number of reduced-volume cartridges are disclosed herein. In the following description, numerous specific details are set forth in order to provide a thorough understanding of the present invention. However, it will be obvious to one of ordinary skill in the art that the invention may be practiced without these specific details. In other instances, well-known structures and processing steps have not been shown in particular detail in order to avoid unnecessarily obscuring the present invention. Additionally, it should be noted that the invention is applicable to reduce the hold-up container volume in a variety of sizes of medical fluid cartridges used syringes with needles, or used in a pump or injection pen used to administer fluid, gasses and medical fluids to a patient. Any implementation of volume-displacing plunger of the present invention may be configured singularly or separately, and be combined by a number of methods, including but not limited to injection molding, stamping, co-molding, insert molding, extruding, ultrasonic welding, adhesive bonding, interference fit, press fit, friction fit, compression fit, heat welding, a threaded means or the like. It is appreciated, however, that the present invention is not limited to these devices.

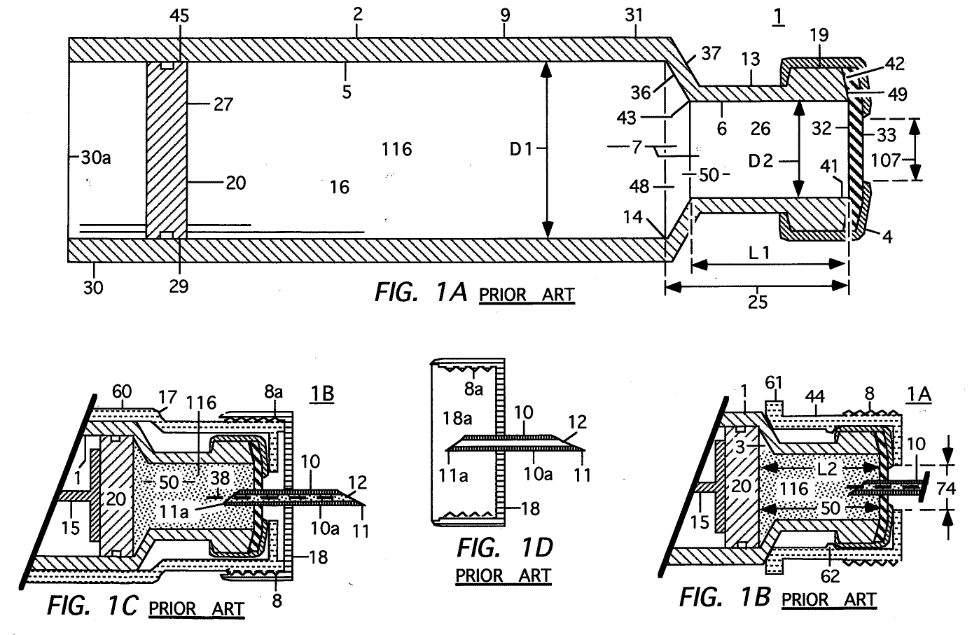

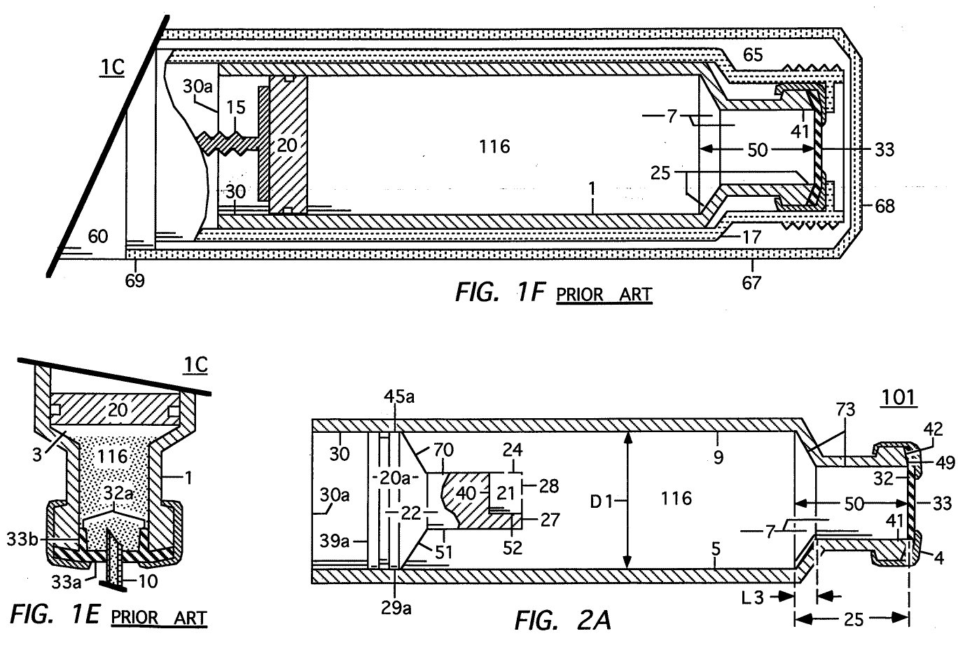

FIG. 1A illustrates a prior art cartridge 1, and FIGS. 1B and 1C illustrates a prior art medical fluid dispensing apparatus 1A, including prior art cartridge 1 of FIG. 1A, as manufactured and sold. FIG. 1E illustrates the prior art medical fluid delivery device, as manufactured and sold, with a cartridge 1, filled with a medical fluid, disposed in a cartridge holder, with a cap covering the cartridge holder.

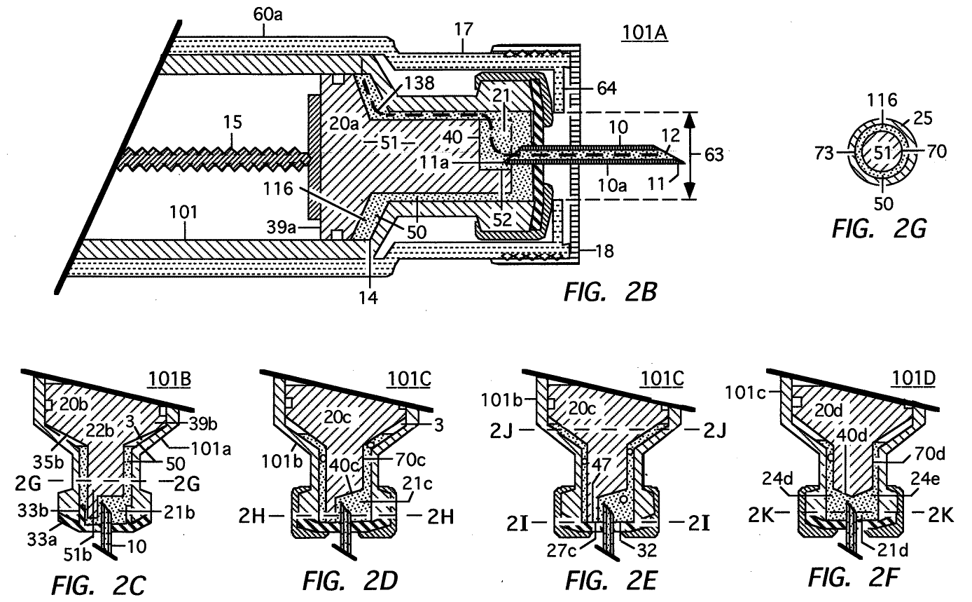

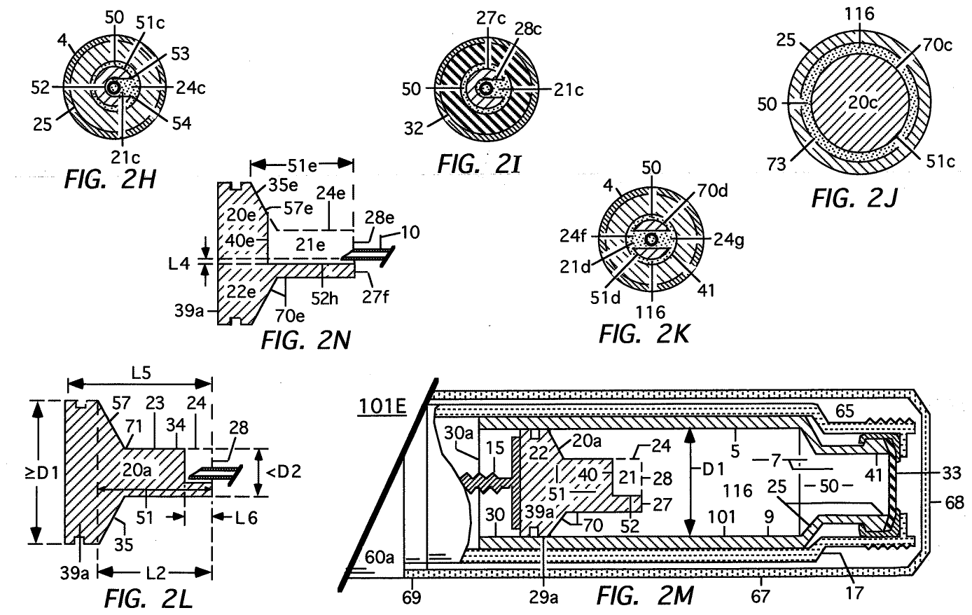

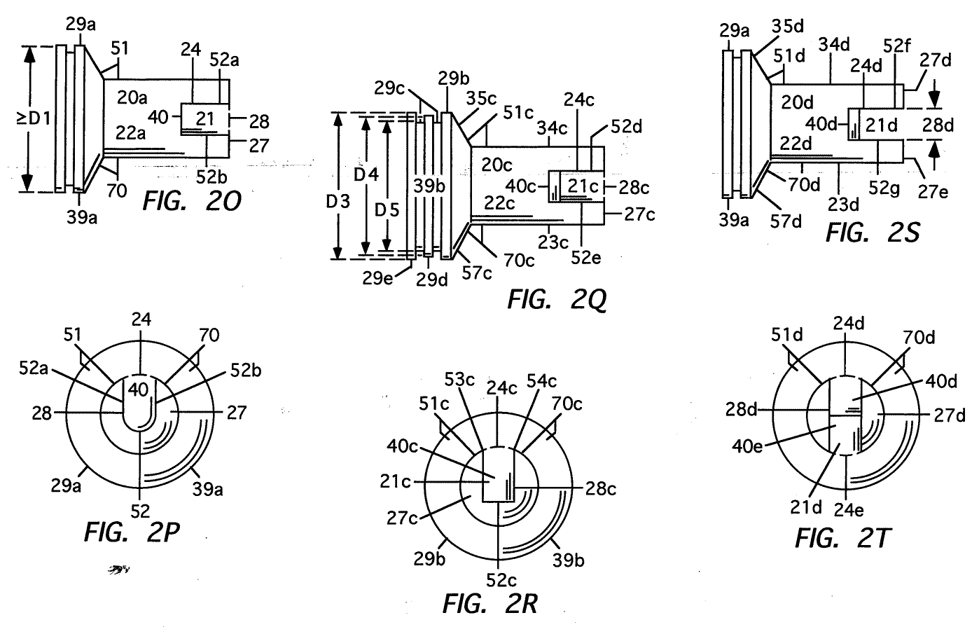

FIG. 1A illustrates a cross-sectional side view of a prior art cartridge 1, comprising a body 9, configured with a proximal elongate tube 2 having an interior cavity 16, with an inside diameter D1, that may be configured to measure 6.85 mm or 0.269″, 8.65 mm or 0.340″, 9.25 mm or 0.368″, or 9.65 mm or 0.368″ per ISO 13926-1:2018, defined by interior wall 5, and a proximal portion 30 with an open proximal end or opening 30a, and a distal portion 31. Distal portion 31 comprises a funneled hollow distal end 25, configured in a funnel shape and includes a first frustoconical conical portion 37, that may include an arcuate profile, joined with second hollow duct or narrow tube or portion 13 with an open distal end 41, that may be configured to measure 3.15 mm or 0.124″ per ISO 13926-1:2018. Conical portion 37, having an axial length of L3, as shown in FIG. 2A, includes an interior cavity 48, defined by interior wall 36, in communication with an interior cavity 26, of the narrow portion 13 configured with a short interior wall 6 with an inside diameter D2. Interior wall 36 is configured between the corner bend 14, of the elongate tube 2, and the corner bend 43 of the narrow portion 13. The interior cavity 26, of the narrow portion 13, may be configured with an axial length of L1, that may be configured to measure 5 mm or 0.197″ per ISO 13926-1:2018, configured along the short interior wall 6, from the distal end wall 49 of the funneled hollow distal end 25, to the corner bend 43 in the narrow portion 13. A first interior wall 5, of elongate tube 2, intersects with a second interior wall 36, of conical portion 37, that intersects with a third short interior wall 6 of hollow duct 13. The interior cavity 16, of body 9, in communication with the interior cavity 48, of conical portion 37, in communication with the interior cavity 26, of the narrow portion 13, combine to form a hollow barrel 7 for accommodating fluid or gas 116.

Interior cavity 48, of conical portion 37, and interior cavity 26 of narrow portion 13 combine form a funnel-shaped interior cavity 50, with an axial length L2 as shown in FIG. 1B, in the funneled hollow distal end 25. The interior cavity 50 is configured between the bend 14, of the elongate tube 2, and the distal end wall 49 of the funneled hollow distal end 25. The prior art plunger 20, may be configured per ISO 13926-2, as used in the current injection pens, do not displace any significant portion of the funneled interior cavity 50, that is characterized as a deadspace cavity. Funneled interior cavity 50 includes a cubic volumetric measure of approx. 0.15 ml, or 30% of a 0.5 ml dose. The deadspace cavity may include at least a portion of the interior cavity 16 of the proximal elongate tube 2, should the plunger 20 fail to advance during use to the corner bend 14 of the elongate tube 2.

The manufacturing process to produce the hollow barrel 7 of the cartridge 1 or a syringe has a geometric dimensional variability due to natural manufacturing tolerances. This geometric dimensional variability in the fluid path of the hollow barrel 7 between the plunger 20 and membrane 33 is known as “container hold up” volume. Any air or air bubbles 3, trapped in the hollow barrel 7 with the fluid 116, play a significant role in calculating the amount of fluid added to the hollow barrel 7 to ensure the accuracy of the intended single-dose or multi-dose injection volume. The deadspace configured in the funneled interior cavity 50 of the funneled hollow distal end 25 of the cartridge 1 must be added the injection volume calculations resulting in additional over-fill of fluid 116 in the hollow barrel 7. The hollow barrel 7 must be intentionally over-filled to ensure the minimum possible dose, or doses are delivered to the patient to avoid systemic under-dosing. The current state of fluid over-fill limits the number of units the current production lot volumes can fill and increases the cost of each unit. The commercialization of the reduced-volume cartridges 101, 101a-101c, 201 and 301 of the present invention will increase the number of cartridges 1 or vials that can be produced from existing production lot volumes, helping to mitigate supply-chain restraints and positively impact patients who are at times forced to go without their medicine due to the ongoing drug shortages. A quick visual comparison can be made between the “container hold up” volume of the hollow barrel 7 in cartridge 1 of FIG. 1F, and the “container hold up” volume of the hollow barrel 7 in the reduced-volume cartridge 101 of FIG. 2A of the present invention.

A pierceable membrane 33, having a proximal facing end wall 32, is disposed to cover the distal opening 41 of the funneled hollow distal end 25, and may be secured by a cap 4 crimped over a lip 19 of the narrow portion 13. Cap 4 includes an opening 107, shown between arrows, allowing the membrane 33 to be pierced by a proximal end 11a of a needle 10, as shown in FIG. 1C. The proximal facing end wall 32, of membrane 33, may be attached by an adhesive to the distal end wall 49 of the funneled hollow distal end 25, eliminating the need for the cap 4 to secure the membrane 33 to the distal end wall 49. The proximal portion 30 of the body 9 includes a moveable plunger 20, disposed in the elongate tube 2, that may be configured with at least one protrusion or radial ring 29, having an outside diameter exceeding the inside diameter D1 of the elongate tube 2, configured to form at least one liquid-tight and air-tight seal 45, may be configured per ISO 13926-3, with an interior wall 5 of the elongate tube 2 of body 9. Plunger 20 includes a distal end wall 27, that may include a slight distal taper or crown. The outside diameter D1 of the solid proximal ends 39a, 139 and 239 or the at least one radial ring 29, 129, and 229 of the plunger 20 and the volume-displacing plungers 20a, 20b, 20c, 20d, 120, 120a, and 220 of the present invention may be configured to measure between 6.6 mm and 7.1 mm for a cartridge with an inside diameter of 6.85 mm, between 8.4 mm and 8.9 mm for a cartridge with an inside diameter of 8.65 mm, between 9.0 mm and 9.5 mm for a cartridge with an inside diameter of 9.25 mm, and between 9.4 mm and 9.9 mm for a cartridge with an inside diameter of 9.65 mm per ISO 13926-2:2018.

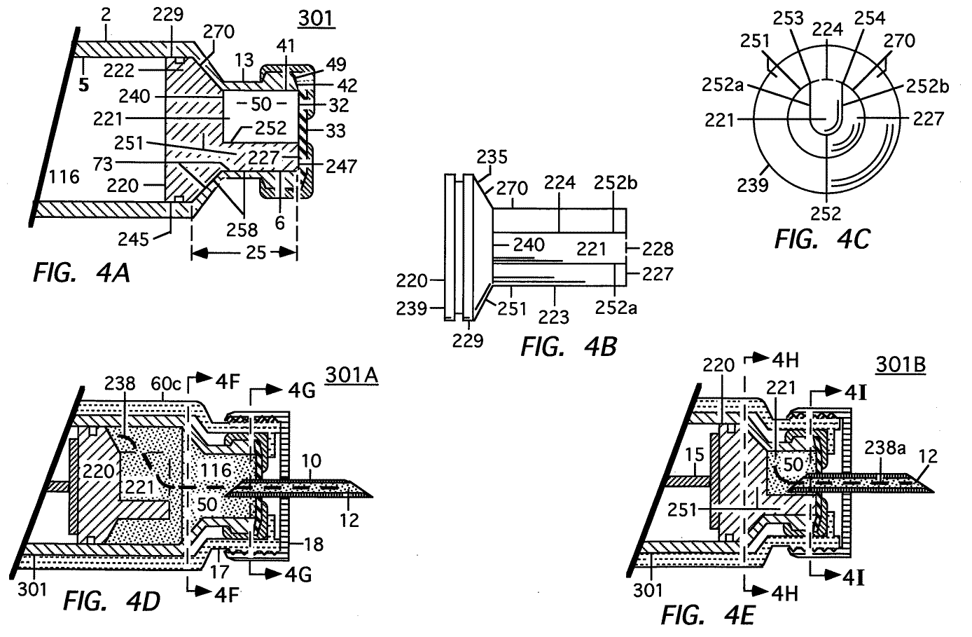

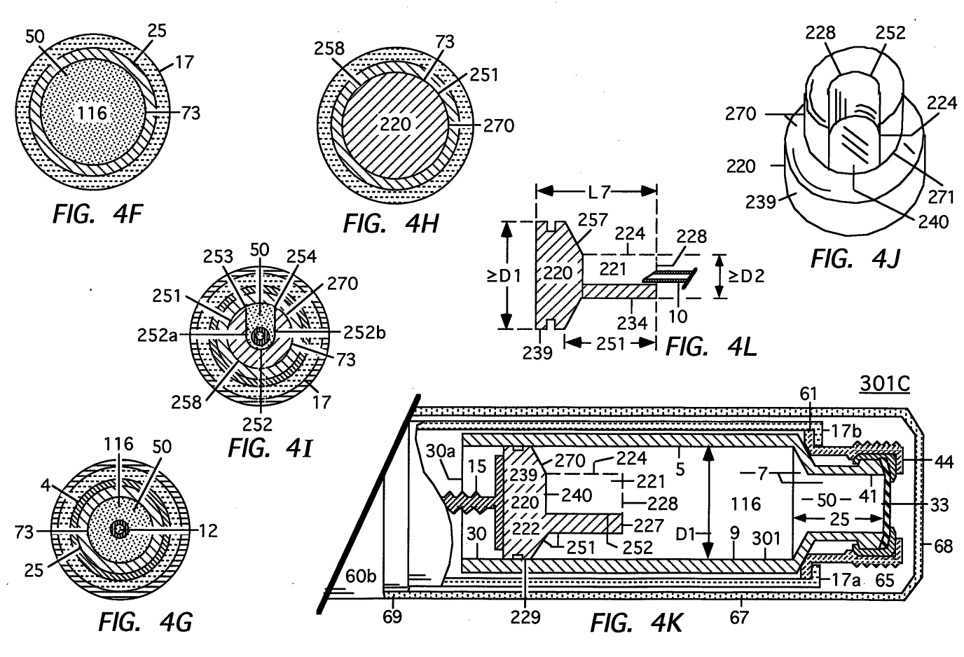

FIG. 1B illustrates a cross-sectional side view of a prior art cartridge 1A with a screw cap 44 disposed over the cap 4, as shown in FIG. 1A. Undispensed fluid 116 is retained in the funneled interior cavity 50 of the funneled hollow distal end 25 as shown in FIG. 1A, after the plunger 20 is moved to the distal end of elongate portion 2 of the hollow barrel 7 as shown in FIG. 1A. A screw cap 44, that may be color coded is secured over the cap 4 as shown in FIG. 2A, by an interior lip 62. The screw cap 44, with exterior threads 8, is configured with a proximal annular exterior lip 61 for engaging a stop 17b in a cartridge holder 17a, as shown in FIG. 4K, and an open end 74 to allowing access for a needle 10 to pierce the membrane 33, as shown in FIG. 1A, when a needle hub 18 is attached to the cartridge holder 17a as shown in FIG. 1C. The funneled interior cavity 50, of the funneled hollow distal end 25, comprises an axial length of L2. The elongate tube 2 of body 9 includes an interior wall 5 with an inside diameter D1, as shown in FIG. 1A, exceeding the inside diameter D2 of the short interior wall 6 of narrow tube 13, to allow air 3, trapped in the hollow barrel 7, as shown in FIG. 1A, to migrate away from the needle 10 as the fluid 116 is dispensed from the hollow barrel 7 through the lumen 12 of the needle 10 as shown in FIG. 1C. The funneled interior cavity 50 comprises an axial length of L2, configured between the proximal end wall 32, of the membrane 33, and the bend 14 of body 9 as shown in FIG. 1A.

FIG. 1C illustrates a cross-sectional side view of a prior art medical fluid dispensing apparatus 1B of a dispensing device 60 with fluid 116 remaining within the funneled interior cavity 50 of the cartridge 1, after the plunger 20 is moved to the distal end of the hollow barrel 7, as shown in FIG. 1A. The cartridge 1 is oriented in the horizontal position routinely used to inject the peri-umbilical area of the abdomen and outer aspect of the arms by a dispensing device 60, as shown in 1B. The medical fluid dispensing device 60 includes a cartridge holder 17 with distal open end 63, and a stop 64, as shown in FIG. 2B, that may include threads 8, selectively coupled to a needle hub 18, that may include matching threads 8a, comprising a needle 10 with a rigid exterior wall 10a, a sharpened proximal end 11a and sharpened distal end 11 with a hollow lumen 12 configured there between. The dispensing device 60 or a syringe with any of the reduced-volume cartridges of the present invention may be used to dispense an injection or coupled to a needle hub or I.V. set by a number of methods, including but not limited to a slip-fit, a luer lock-fit, a press-fit, a compression-fit, a friction-fit, or the like. Sharpened proximal end 11a of needle 10, having pierced through the membrane 33, as shown in FIG. 1A, is disposed within the funneled interior cavity 50 of the funneled hollow distal end 25, as shown in FIG. 1A, allowing fluid or gas 116 to flow from the hollow barrel 7, as shown in FIG. 1A, through the needle lumen 12. When the push rod 15 and plunger 20 are advanced, translated or moved to the distal portion 31 of the hollow barrel 7, as shown in FIG. 1A, the majority of fluid 116 previously disposed in interior cavity 16, as shown in FIG. 1A, has been dispensed through the funneled interior cavity 50, forming a substantially-linear flow path 38 within the lumen 12 of needle 10. A measurable portion of drug wastage fluid 116 remains within the interior cavity 50, and is discarded with the used cartridge 1 into the waste stream. As shown in FIG. 1A, the funneled interior cavity 50 of the funneled hollow distal end 25, is closed by proximal facing wall 32 of the membrane 33.

During the filling process of the hollow barrel 7, as shown in FIG. 1A, a small amount of air 3 may remain along with the fluid 116, inside the hollow barrel 7. Air 3, as shown in FIG. 1B, may also form from the fluid 116 disposed within the hollow barrel 7, as shown in FIG. 1A, when a cartridge 1 containing insulin warms after it is removed from refrigeration. Any air or air bubbles 3, as shown in FIG. 1B, contained within the hollow barrel 7 as shown in FIG. 1A, must be removed or separated from the fluid 116 before the dose is dispensed through the lumen 12 of the needle 10. Since air 3, as shown in FIG. 1B, rises upward in fluid 116, the air 3 within the hollow barrel 7 gravitates towards the corner bend 14 of the body 9 as shown in FIG. 1A, keeping any air 3 a safe distance from the proximal end 11a of the needle 10. With the prevalence of ongoing drug shortages, patients are utilizing a needle/syringe to harvest the medical fluid remaining in the deadspace of funneled interior cavity 50 directly from the cartridge 1, by advancing the syringe's needle tip through the membrane 33 and withdrawing as much medical fluid 116 as possible. After the medical fluid 116 is harvested from the funneled interior cavity 50 from the first cartridge 1, the medical fluid 116 can be transferred into a second cartridge 1, or combined with medical fluid 116 to create another dose from the syringe.

FIG. 1D illustrates a cross-sectional side view of a prior art needle hub 18, configured with a hollow interior cavity 18a including interior threads 8a, with a needle 10, with a rigid exterior wall 10a bonded to the needle hub 18, a sharpened proximal end 11a, and sharpened distal end 11 with a hollow lumen 12 configured there between.

FIG. 1E illustrates a prior art cartridge/needle apparatus 1C, comprising the prior art cartridge 1, with the proximal end 11a, as shown in FIG. 1C, of the needle 10, disposed in the fluid 116 in the interior cavity 50, as shown in FIG. 1C, in the vertical orientation routinely used to inject the anterior aspect of the thighs when the patient is seated. The air 3 within the hollow barrel 7 gravitates towards the distal end wall 27 as shown in FIG. 1A, of the plunger 20 when the plunger 20 is disposed at the distal end of the interior cavity 16 of the elongate tube 2, as shown in FIG. 1A, keeping any air 3 a safe distance from the proximal end 11a, as shown in FIG. 1C, of the needle 10. The membrane 33a may be configured with a hollow collar 33b, with proximal end wall 32a, sealingly engaging the short interior wall 6 of the narrow tube 13 as shown in FIG. 1A, securing the membrane over the open end 41 of the funneled hollow distal end 25 as shown in FIG. 1A.

FIG. 1F illustrates a prior art medicament delivery apparatus 1C, as manufactured and sold, comprising a fluid dispensing device 60, comprising a cartridge holder 17 holding a cartridge 1 with a hollow barrel 7 containing a liquid or gas 116, a screw 15 disposed in the cartridge for moving a plunger 20 in the hollow barrel 7, a cap 67 with a closed distal end 68 and an elongate interior cavity 65 with a proximal open end 69 attached to the fluid dispensing device 60, the cartridge 1 including a hollow barrel 7 with a hollow proximal portion 30 with an open proximal end 30a and a funneled hollow distal end 25 with a funneled interior cavity 50 with an open distal end 41, a membrane 33 covering the open distal end 41 of the funneled hollow distal end 25, and a plunger 20 disposed inside the hollow barrel 7 through the open proximal end 30a of the hollow barrel 7 available for accommodating a liquid or gas 116.

FIGS. 2A, 2B and 2M illustrate the reduced-volume cartridges 101, 101 a, 101 b and 101c of the present invention, comprising a hollow barrel 7, available for accommodating liquid or gas 116, with volume-displacing plungers 20a, 20b, 20b, 20c, 20d and 20e of the present invention, moveable or translatable within the hollow barrel 7. Volume-displacing plungers 20a, 20b, 20b, 20c, 20d and 20e are configured to reduce the cubic volumetric capacity or measure of fluid or gas 116 within the hollow barrel 7, and the funneled interior cavity 50 of the funneled hollow distal end 25 of the hollow barrel 7, before, during and after the fluid or gas 116 is filled into, or removed from the hollow barrel 7. The reduced-volume cartridges 101, 101a-101c, 201 and 301 of the present invention may be configured with the specifications recommended by the ISO 13926-1:2018 standards. The profile of the interior wall 36, of the conical portion 37 of the hollow barrel 7 of the reduced-volume cartridges 101, 101a-101c, 201 and 301 of the present invention may be configured with an arcuate profile. A reducing arcuate exterior wall maybe substituted for the exterior profile of the conical portions 57, 57a, 57b, 57c, 57d, 157 and 257 of the exterior walls 70, 70a, 70b, 70c, 70d, 70 e, 170, 170 a, and 270 of the distal ends 51, 51 a, 51 b, 51 c, 51 d, 51 e, 151 and 251 respectively, of the all the volume-displacing plungers of the present invention. A substantially matching reducing arcuate interior wall portion maybe substituted for the interior profile of the interior wall 36 of the conical portion 37 of the body 9 of the medical fluid cartridges 101, 101a-101c, 201 and 301 of the present invention. The axial length L2 of the funneled distal ends 51, 51a, 51b, 51c, 51d, 51e, 151, 151a and 251 of the present invention varies due to the manufacturing variability of the axial length L3 of the conical portions 57, 57 a, 57 b, 57 c, 57 d, 57 e, 157 and 257 and the inside diameters D1 of 4 different cartridges per ISO 13926-1:2018. The elastomeric volume-displacing plungers 20a, 20b, 20c, 20d, 20e, 120, 120a, and 220 of the present invention may comprise butyl-based formulations, including, but not limited to chlorobutyl and bromobutyl elastomers, and may be coated with a fluoropolymer spray coating or film or other formulations to reduce or eliminate any extractable and leachable materials in the plungers from interacting with the drugs in the cartridge. Formulations are also either added directly to the plungers or applied to the exterior to improve the break force and glide force profiles between at least one exterior wall of the plunger and an interior wall of a cartridge.

The proximal portions 39a, 39b, 139 and 239 of the elastomeric volume-displacing plungers 20a, 20b, 20c, 20d, 20e, 120, 120a, and 220 of the present invention may be configured per ISO 13926-2 with multiple outside diameters D3, D4 and D5 as shown in FIG, 2Q. Each proximal portion 39a, 39b, 139 and 239 may be configured with a medial radial ring 29d with an outside diameter D4, configured between substantially matching radial rings 29c with an outside diameter D5, configured between substantially matching radial rings 29b and 29e with an outside diameter D3. As shown in FIG. 2Q, the outside diameter D3 of radial ring 29b and radial ring 29e are configured to measure a minimum of 7.1 mm or 0.279 in. for glass cylinder having an inside diameter 6.85 mm. The outside diameter D4 of radial ring 29d is configured to measure a minimum of 7.0 mm or 0.275 in. for glass cylinder having an inside diameter 6.85 mm. The outside diameter D5 of radial ring 29c is configured to measure a minimum of 6.0 mm or 0.236 in. for glass cylinder having an inside diameter 6.85 mm. As shown in FIG. 2Q, the outside diameter D3 of radial ring 29b and radial ring 29e are configured to measure a minimum of 8.9 mm or 0.350 in. for glass cylinder having an inside diameter 8.65 mm. The outside diameter D4 of radial ring 29d is configured to measure a minimum of 8.8 mm or 0.346 in. for glass cylinder having an inside diameter 8.65 mm. The outside diameter D5 of radial ring 29c is configured to measure a minimum of 8.4 mm or 0.330in. for glass cylinder having an inside diameter 8.65 mm. As shown in FIG. 2Q, the outside diameter D3 of radial ring 29b and radial ring 29e are configured to measure a minimum of 9.5 mm or 0.374 in. for glass cylinder having an inside diameter 9.25 mm. The outside diameter D4 of radial ring 29d is configured to measure a minimum of 9.4 mm or 0.370 in. for glass cylinder having an inside diameter 9.25 mm. The outside diameter D5 of radial ring 29c is configured to measure a minimum of 9.0 mm or 0.354in. for glass cylinder having an inside diameter 9.25 mm. As shown in FIG. 2Q, the outside diameter D3 of radial ring 29b and radial ring 29e are configured to measure a minimum of 9.9 mm or 0.390 in. for glass cylinder having an inside diameter 9.65 mm. The outside diameter D4 of radial ring 29d is configured to measure a minimum of 9.8 mm or 0.386 in. for glass cylinder having an inside diameter 9.65 mm. The outside diameter D5 of radial ring 29c is configured to measure a minimum of 9.4 mm or 0.370 in. for glass cylinder having an inside diameter 9.65 mm.

According to one implementation of the present invention FIG. 2A, illustrates a cross-sectional side view of the reduced-volume cartridge 101 of the present invention, configured with a volume-displacing plunger 20a disposed a hollow barrel 7, reducing the cubic volumetric capacity in the hollow barrel 7 available for accommodating liquid or gas 116. The reduced-volume cartridge 101 is configured with a hollow barrel 7 with a proximal portion 30, with an open proximal end 30a, joined with a distal portion 31, as shown in FIG. 1A, with a funneled hollow distal end 25 with a funneled interior cavity 50 with an open distal end 41. As shown in FIG. 1A, the hollow barrel 7 is defined by the interior wall 5, joined with the interior conical wall 36, joined with the short interior wall 6. The volume-displacing plunger 20a comprises a body 22, configured with a solid proximal portion 39, that may include at least one exterior protrusion or radial ring 29a, with an outside diameter ≥D1 as shown in FIG. 2L. The at least one radial ring 29a is configured to form a least a first liquid-tight and air-tight seal 45a with the interior wall 5 of elongate tube 2, as shown in FIG. 1A. The body 22, comprises a solid proximal portion 39a, a conical portion 57 and an elongate distal portion 34, as shown in FIG. 2L, and may be configured with a combined axial length of L5, as shown in FIG. 2L. The body 22, may comprise only a solid proximal portion 39a and an elongate portion 34. The interior conical wall 36, of the conical portion 37, as shown in FIG. 1A, and the short interior wall 6 of the narrow portion 13, as shown in FIG. 1A, of the hollow barrel 7, combine to form funneled interior wall 73, configured in the funneled hollow distal end 25.

The body 22, comprises a distal end 51, configured with a conical portion 57, having an exterior conical wall 35, as shown in FIG. 2L, and an elongate distal portion 34 with an axial exterior side wall 23, as shown in FIG. 2L, and a distal end wall 27. When the volume-displacing plunger 20a is disposed in the funneled interior cavity 50, the distal end wall 27 of the funneled distal end 51 is configured to not to engage the proximal wall of 32 of the membrane 33. The exterior conical wall 35 and the axial exterior wall 23, of the distal end 51, are configured to form funneled exterior wall 70, with a bend 71, as shown in FIG. 2L. The funnel-shaped exterior profile of the funneled exterior wall 70 of the distal end 51 of the body 22, is configured to remain separated from the funnel-shaped interior profile of the funneled interior wall 73, of the funneled interior cavity 50 of the funneled hollow distal end 25. The funneled distal end 51, includes a distal interior cavity 21, configured with a first opening 28, shown as a broken line, configured in a distal end wall 27, and a second opening 24, shown as a broken line, configured in the funneled exterior wall 70. The distal interior cavity 21 is defined by a solid proximal interior wall 40 and an opposing opening 28 in the distal end wall 27, the solid proximal interior wall 40 is configured between a least one axial interior side wall 52 and at least one opening 24 in at least one funneled exterior wall 70. The first opening 28 and the distal interior cavity 21 are configured to receive a hollow needle 10, as shown in FIG. 2B. The first opening 28 may be configured to intersect with the second opening 24. The open end or opening 41, of the funneled hollow distal end 25, is covered with a pierceable membrane 33, that may be configured with a radial side wall configured to surround and engage the lip 19, as shown FIG. 1A of the funneled hollow distal end 25. The membrane 33 may comprise a multi-compound elastomeric rubber liner configured to withstand multiple piercings and maintain a leak-proof profile. A second liquid-tight and air tight seal 42, is configured between a portion of the proximal facing wall 32, of the membrane 33, and the distal end wall 49 of the funneled hollow distal end 25. The solid proximal interior wall 40, intersects the proximal end of interior axial side wall 52, and is configured to extend to the funneled exterior wall 70 of the distal end 51.

A medical fluid cartridge 101 comprising a body 9 including a hollow barrel 7 with a hollow proximal portion 30 with an interior wall 5 with a first inside diameter D1 with an open proximal end 30a and a funneled hollow distal end 25 with an funneled interior cavity 50 with a distal open end 41, a membrane 33 covering the distal open end 41 of the funneled hollow distal end 25, and a volume-displacing plunger 20a disposed inside the hollow barrel 7 through the open proximal end 30a of the hollow proximal portion 30 and configured to reduce the cubic volumetric capacity inside the hollow barrel 7 available for accommodating a liquid or gas 116, the volume displacing plunger 20a including a body 22 with a proximal portion 39a with an exterior wall 29a with a first outside diameter equal to or exceeding the first inside diameter D1 of the interior wall 5 of the hollow proximal portion 30 and a funneled distal end 51 with a distal interior cavity 21 defined by a solid proximal interior wall 40 and an opposing opening 28 in a distal end wall 27, the solid proximal interior wall 40 configured between a least one axial interior side wall 52 and at least one opening 24 in at least one funneled exterior wall 70.

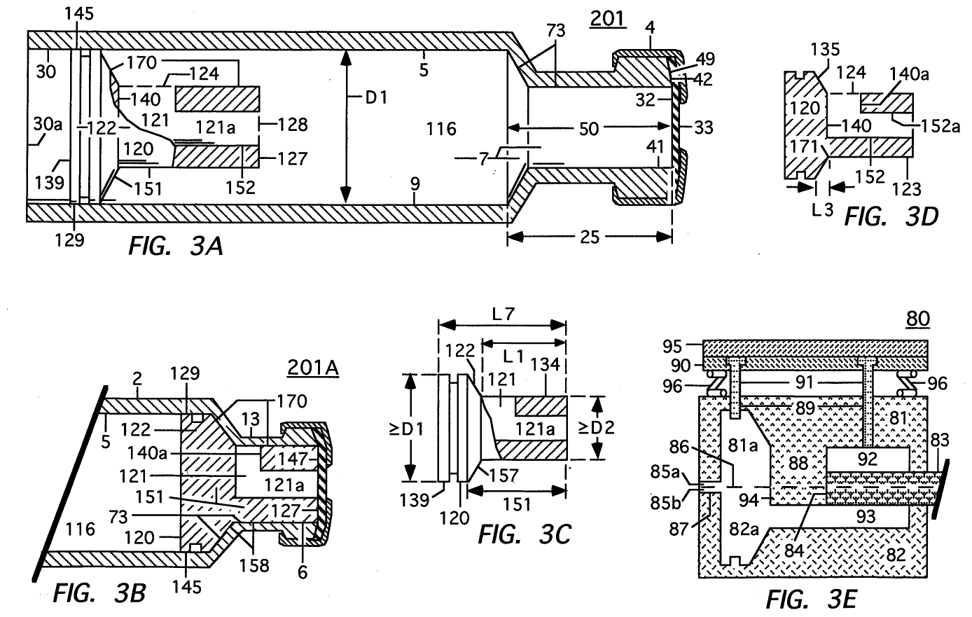

FIG. 2B illustrates a cross-sectional side view of the medical fluid dispensing apparatus 101A of the present invention with the reduced-volume cartridge 101 disposed in the cartridge holder 17 of dispensing device 60a. The dispensing device 60a is attached to a needle hub 18, with the majority of the fluid 116 emptied from the hollow barrel 7, as shown in FIG. 2A, by a pushrod or screw 15 moving the volume-displacing plunger 20a through the hollow barrel 7, as shown FIG. 2A, and into the funneled interior cavity 50, of the funneled hollow distal end 25, as shown in FIG. 2A. The proximal end 11a, of the needle 10, has pierced the membrane 33, as shown in FIG. 1C, and is disposed in the funneled interior cavity 50 of the funneled hollow distal end 25, as shown in FIG. 2A. The dispensing device 60a comprises the cartridge holder 17, configured with an open distal end 63, shown between arrows, with a stop 64, for securing the reduced-volume cartridge 101. The needle 10 may be configured with a sharpened distal end 11, or configured with a needle with a bend and a sharpened distal end, or a needle with a sharpened distal end, configured to retract into the hollow barrel of a syringe, or attached to a hollow tube in a pump, or to an infusion line. The distal end 51 of the volume-displacing plunger 20a, has displaced the majority of the liquid and gas 116 previously stored within the interior cavity 50, of the funneled hollow distal end 25, as shown in FIG. 2A. The elongate distal portion 34, as shown in FIG. 2L, of the distal end 51, is configured with an outside diameter <D2, and an axial length shorter than L1, as shown in FIG. 3C. The funneled exterior wall 70, as shown in FIG. 2A, of the distal end 51, is spaced apart from the funneled interior wall 73, of the funneled hollow distal end 25, as shown in FIG. 2A, within interior cavity 50. When the volume-displacing plunger 20a is disposed in the funneled interior cavity 50 of the funneled hollow distal end 25, as shown in in FIG. 2A, the distal interior cavity 21 includes a solid proximal interior wall 40 sufficiently spaced from the sharpened proximal end 11a of the needle 10, as shown in FIGS. 2C and 2L, without the sharpened proximal end 11a engaging the solid proximal interior wall 40. The distal interior cavity 21 includes at least one axial interior side wall 52 sufficiently spaced from the rigid exterior wall 10a of the needle 10, without the rigid exterior wall 10a engaging the axial interior side wall 52. The distal end 51 comprises a distal interior cavity 21, in communication with the interior cavity 50, of the funneled hollow distal end 25, as shown in FIG. 2A, in communication with the needle lumen 12 of the needle 10. A flow path 138 is configured between the hollow barrel 7, as shown in FIG. 2A, into and through the interior cavity 50, now defined between the funneled interior wall 73 of the funneled hollow distal end 25 and the funneled exterior wall 70, as shown in FIG. 2A, of the funneled distal end 51, into and through the distal interior cavity 21 of the funneled distal end 51, and into and through the needle lumen 12. The solid proximal portion 39a, of the volume-displacing plunger 20a, is configured to stop at the bend 14 of the body 9, as shown in FIG. 1A, maintaining a space in the interior cavity 50, between the elongate axial portion 34, as shown in FIG. 2L, of the funneled exterior wall 70 of the volume-displacing plunger 51, and the short interior wall 6, as shown in FIG. 1A, of the funneled interior wall 73 of the funneled hollow distal end 25 as shown in FIG. 2A, to allow any residual air or air bubble 3, as shown in FIG. 1C, in the fluid 116 in flow path 138, to remain at a safe distance from the proximal end of the needle 11a, as shown in FIG. 1C. The volume-displacing plungers, 20a, 20b, 20c, 20d and 20e of the present invention include comprise a proximal end 39a with an exterior wall or radial ring 29a, configured to conform to meet the specifications of the ISO 13926-2:2018 and ISO 13926-3:201a. The funneled exterior walls 70, 70a, 70c, 70d and 70e of the volume-displacing plungers, 20a, 20b, 20c, 20d and 20e of the present invention are sized and configured not to engage the funneled interior walls 73 of the reduced-volume cartridges 101, 101a, 101b and 101c of the present invention, eliminating the need to establish a standard or test the seal integrity of the volume-displacing plungers of the present invention.

A medical fluid dispensing apparatus 101A comprising a medical fluid delivery device 60a including a cartridge holder 17 holding a reduced-volume cartridge 101 with a hollow barrel 7 containing a liquid or gas 116, a screw 15 disposed in the reduced-volume cartridge 101 for moving a volume-displacing plunger 20a in the hollow barrel 7, a needle hub 18 with a needle 10 with a rigid exterior wall 10a with a sharpened proximal end 11a and sharpened distal end 11 with a hollow lumen 12 configured there between, the needle hub 18 including a hollow interior cavity 18a attached to a distal open end 63 of the cartridge holder 17, a cartridge 101 including a hollow barrel 7 with a hollow proximal portion 30 with an interior wall 5 with a first inside diameter D1 with an open proximal end 30a and a funneled hollow distal end 25 with an funneled interior cavity 50 with an open distal end 41, a membrane 33 covering the open distal end 41 of the funneled hollow distal end 25 and the sharpened proximal end 11a of the needle 10 disposed in the funneled interior cavity 50 through the membrane 33, and a volume-displacing plunger 20a disposed through the open proximal end 30a of the hollow proximal portion 30 and configured to reduce the cubic volumetric capacity inside the hollow barrel 7 available for accommodating a liquid or gas 116, the volume displacing plunger 20a including a body 22 with a solid proximal portion 39a and an exterior wall 29 with a first outside diameter equal to or exceeding the first inside diameter D1 of the interior wall 5 of the hollow proximal portion 30a and a funneled distal end 51 having a distal interior cavity 21 defined by a solid proximal interior wall 40 and an opposing opening 28 in a distal end wall 27, the solid proximal interior wall 40 configured between a least one axial interior side wall 52 and at least one opening 24 in at least one funneled exterior wall 70.

FIG. 2C is a cross-sectional side view illustrating the reduced-volume cartridge/needle apparatus 101B of the present invention, with the reduced-volume-cartridge 101a oriented in a vertical position, with the volume-displacing plunger 20b and the needle 10, disposed in the funneled interior cavity 50 of the funneled hollow distal end 25, as shown in FIG. 2A. The funneled distal end 51b of the volume-displacing plunger 20b, comprises a distal interior cavity 21b, in communication with the funneled interior cavity 50, of the funneled hollow distal end 25, as shown in FIG. 2A, in communication with the needle lumen 12 as shown in FIG. 2B, of the needle 10. Air 3, that may reside within the funneled interior cavity 50, has gravitated through the fluid 116, as shown in FIG. 2B, toward the corner bend 14 in the conical portion 37, as shown in FIG. 1A, maintaining any air 3 within the funneled interior cavity 50 at a safe distance from the proximal end 11a of the needle 10, as shown in FIG. 2B, disposed in the distal interior cavity 21b. To allow any air or air bubbles 3, that may be contained within the funneled interior cavity 50 and the fluid 116, as shown in FIG. 2B, to gravitate away from the needle lumen 12, as shown on FIG. 2B, a space is configured within the funneled interior cavity 50, between the funneled exterior wall 70b, of the distal end 51b, and the funneled interior wall 73, of the funneled hollow distal end 25, as shown in FIG. 2A. The solid proximal portion 39b of the body 22b engages and stops at the bend 14 of the body 9 as shown in FIG. 1A, maintaining a space in the funneled interior cavity 50 between the distal end wall 27b of the funneled distal end 51b, as shown in FIG. 2A, and the proximal facing wall 32 of the membrane 33 as shown in FIG. 2A. Volume displacing plungers 20a, 20c, 20d, 20e, 120, 120a, 220 and 220 of the present invention, may also comprise a solid proximal portion 39a, 139 or 229, configured to stop at the corner bend 14, of the body 9, as shown in FIG. 1A.

FIG. 2D is a cross-sectional side view illustrating the reduced-volume cartridge needle apparatus 101C of the present invention, with the reduced-volume-cartridge 101b in a vertical position, with a volume-displacing plunger 20c with the distal end 51c, as shown in FIG. 2Q, and the needle 10 disposed within the funneled interior cavity 50 as shown in FIG. 2C, of the funneled hollow distal end 25 as shown in FIG. 2A. The distal end 51c, as shown in FIG. 2Q, includes an elongated distal portion 34c, as shown in FIG. 2Q, of the funneled exterior wall 70c, and a distal interior cavity 21c, defined by an angled solid proximal interior wall 40c, configured to channel any air or air bubbles 3 from the fluid 116 as shown in FIG. 2B, disposed in funneled interior cavity 50 as shown in FIG. 2C, away from the proximal end 11a of the needle 10 as shown in FIG. 2B. The distal end 51c, as shown in FIG. 2Q, comprises a distal interior cavity 21c in communication with the interior cavity 50, as shown in FIG. 2C, of the funneled hollow distal end 25 as shown in FIG. 2A, in communication with the needle lumen 12 and needle 10 as shown in FIG. 2B.

FIG. 2E is a cross-sectional side view illustrating the reduced-volume cartridge/needle apparatus 101C of FIG. 2D, with the reduced-volume cartridge 101b containing fluid 116, as shown in FIG. 2B, and air 3, as shown in FIG. 2D, oriented in a vertical position. The distal end 51c, as shown in FIG. 2Q, of the volume-displacing plunger 20c, comprises a distal end wall 27c, configured to engage the proximal facing wall 32 of the membrane 33, as shown in FIG. 2A. A liquid-tight and air-tight seal 47 is configured between the distal end wall 27c, of the distal end 51c, as shown in FIG. 2Q, and the proximal facing wall 32 of the membrane 33 as shown in FIG. 2A.

FIG. 2F is a cross-sectional side view illustrating the reduced-volume cartridge/needle apparatus 101D of the present invention, with the reduced-volume-cartridge 101c, oriented in a vertical position. The volume-displacing plunger 20d, is disposed in the interior cavity 50, as shown in FIG. 2C, of the funneled hollow distal end 25 as shown in FIG. 2A. The distal end 51d, as shown in FIG. 2S, of the volume-displacing plunger 20d, is configured with a distal interior cavity 21d defined by a first sloped solid proximal interior wall 40d and a second sloped solid proximal interior wall 40e, as shown in FIG. 2T, configured to channel any air or air bubbles 3, as shown FIG. 2D, within the fluid 116, as shown FIG. 2B, away from the proximal end 11a of the needle 10 as shown in FIG. 2B. Interior cavity 21d is configured between a first opening 28d, configured between a first distal end wall 27d and a second opposing distal end wall 27e as shown in FIG. 2S, and a second opening 24d and a third opposing opening 24e, each configured in a funneled exterior wall 70d of the distal end 51d as shown in FIG. 2T. The distal end 51d comprises the distal interior cavity 21d, as shown in FIG. 2S, in communication with the interior cavity 50, as shown in FIG. 2C of the funneled hollow distal end 25, as shown in FIG. 2A, in communication with the lumen 12 of the needle 10 as shown in FIG. 2B. The distal end 51d, as shown in FIG. 2S, includes at least one distal end wall 27d that may be configured to engage the proximal side wall 32 of the membrane 33, as shown in FIG. 2A, when the distal end 51d is disposed in the funneled interior cavity 50, as shown in FIG. 2C.

FIG. 2G illustrates a cross-sectional front view of the reduced-volume cartridge/needle apparatus 101B of FIG. 2C, in axis 2G-2G, with the distal end 51b, of volume-displacing plunger 20b as shown in FIG. 2C, displacing the majority of fluid 116 previously disposed in the funneled interior cavity 50 of the funneled hollow distal end 25. The distal end 51b, of the volume-displacing plunger 20a as shown in FIG. 2C, is configured to reduce the space within funneled interior cavity 50 configured to accommodate liquid or gas 116, now defined between the funneled exterior wall 70, of the funneled distal end 51b, and the funneled interior wall 73 of the funneled hollow distal end 25.

FIG. 2H illustrates a cross-sectional front view of the reduced-volume cartridge/needle apparatus 101C of FIG. 2D, in axis 2H-2H, with the distal end 51c, of volume-displacing plunger 20c as shown in FIG. 2D, displacing the majority of fluid 116, as shown in FIG. 2B, previously disposed in the funneled interior cavity 50 of the funneled hollow distal end 25, A cap 4 is configured to secure a pierceable membrane 33 over the distal open end 41 as shown in FIG. 2A, of the funneled hollow distal end 25. The distal end 51c, comprises a distal interior cavity 21c, configured with a sloped or angled solid proximal interior wall 40c, and an opposing first opening 28c as shown in FIG. 2Q, that may be configured with a boxed “U” configuration with a non-radial axial interior side wall 52c in the distal end wall 27c as shown in FIG. 2R. The distal interior cavity 21c is also defined by a first axial interior side wall 52d and an opposing second axial interior side wall 52e, defining a second opening 24c configured in the funneled exterior wall 70c as shown in FIG. 2Q. The second opening 24a, is configured in a portion of the funneled exterior wall 70c, as shown in FIG. 2R, circumscribing the outside periphery of the distal end 51c between a first corner edge 53c and a second corner edge 54c, as shown in FIG. 2R.

FIG. 2I illustrates a cross-sectional front view of the reduced-volume cartridge/needle apparatus 101C of FIG. 2E, in axis 2I-2I, with the distal end 51c, as shown in FIG. 2Q, of the volume-displacing plunger 20c, as shown in FIG. 2E, displacing the majority of fluid 116, as shown in FIG. 2B, previously disposed in the funneled interior cavity 50 of the funneled hollow distal end 25, as shown in FIG. 2A. A liquid-tight and air-tight seal 47, as shown in FIG. 2E, is configured between the distal end wall 27c, of the distal end 51c as shown in FIG. 2Q, and the proximal facing wall 32 of the membrane 33 as shown in FIG. 2A. The distal interior cavity 21c, of the distal end 51c as shown in FIG. 2Q, is in fluid communication with funneled interior cavity 50 of the funneled hollow distal end 25 as shown in FIG. 2A.

FIG. 2J illustrates a cross-sectional front view of the reduced-volume cartridge/needle apparatus 101C of FIG. 2E, in axis 2J-2J, with the conical portion 57c, as shown in FIG. 2Q, of the distal end 51c, of the volume-displacing plunger 20c, displacing a significant portion of the fluid 116 previously disposed in the funneled interior cavity 50 of the funneled hollow distal end 25 of the reduced-volume cartridge 101b as shown in FIG. 2E. Funneled interior cavity 50, may contain air 3 as shown in FIG. 2D, and fluid 116, and is defined by the funneled interior wall 73, of the funneled hollow distal end 25, and the funneled exterior wall 70c of the distal end 51c.

FIG. 2K illustrates a cross-sectional front view of the reduced-volume cartridge/needle apparatus 101C of FIG. 2F, in axis 2K-2K, with the distal end 51d of the volume-displacing plunger 20d, as shown in FIG. 2S, displacing the majority of the fluid 116 previously disposed in the funneled interior cavity 50 of the funneled hollow distal end 25, as shown in FIG. 2A, of the reduced-volume cartridge 101c as shown in FIG. 2F. The distal interior cavity 21d, of the funneled distal end 51d as shown in FIG. 2S, is configured between a first opening 28d, configured between a first distal end wall 27d and a second opposing distal end wall 27e as shown in FIG. 2S, and the third opening 24d and a fourth opposing opening 24e, each shown in a broken line in FIG. 2T, configured in a portion of the funneled exterior wall 70d as shown in FIG. 2T. The distal interior cavity 21d is configured between a first axial interior side wall 52f and a second opposing axial interior side wall 52g as shown in FIG. 2S. A cap 4 is configured to secure a pierceable membrane 33 as shown in FIG. 2A, over the distal open end 41 of the funneled hollow distal end 25 as shown in FIG. 2A.

FIG. 2L, illustrates a cross-sectional side view of the volume-displacing plunger 20a of the present invention comprising the body 22, as shown in FIG. 2A, configured with a solid proximal portion 39a, having an outside diameter ≥D1, a conical portion 57, configured with an exterior conical wall 35, and an elongate distal portion 34 configured with axial exterior side wall 23. The funneled exterior wall 70, as shown in FIG. 2A, of the funneled distal end 51, shown between two arrows, is configured by combining the exterior conical wall 35 and the axial exterior side wall 23. The funneled distal end 51 includes a distal interior cavity 21, shown in FIG. 2O, with a first opening 28 configured in the proximal end wall 27, as shown in FIG. 2O, and a second opening 24, configured in the axial funneled exterior wall 70 as shown in FIG. 2O. The elongate distal portion 34 of funneled distal end 51, is configured with an outside diameter <D2. The interior cavity 21, is defined by a first interior axial side wall 52, that may be configured in a radial or other geometric profile, a second interior axial side wall 52a and a third interior axial side wall 52b as shown in FIG. 2P, and are separated from the rigid exterior wall 10a of the needle 10, as shown in FIG. 2B, by at least 0.001′ or a clearance length of L4 as shown in FIG. 2N, sufficient to keep the rigid exterior wall 10a of the needle 10, as shown in FIG. 2B, from engaging the first interior axial side wall 52, second interior axial side wall 52a and third interior axial side wall 52b, as shown in FIG. 2H. The axial interior side walls 52, 52a and 52b as shown in FIG. 2P, of the distal end 51, are configured in the distal interior cavity 21, at an axial length L6, forming a space between the proximal end 11a of the needle 10, as shown in FIG. 2B, and the solid proximal interior wall 40, as shown in FIG. 2A. The axial length L6, of the interior radial or axial side walls 52, 52a, 52b, 52c, 52d, 52e, 52f and 52g are configured to form a space between the proximal end 11a of a needle 10. as shown in FIGS. 2B and 2C and the proximal interior walls 40, 40a, 40b, 40c, 40d and 40e of the interior cavities 21, 21a, 21b, 21c, 21d and 21e of the volume-displacing plungers 20a, 20b, 20c, 20d, and 20e of the present invention.

FIG. 2M, illustrates a cross-sectional side view of the medical fluid delivery apparatus 101E of the present invention. The medical fluid delivery apparatus 101E comprising, a medical fluid delivery device 60a including a cartridge holder 17 holding a cartridge 101 with a hollow barrel 7 containing a liquid or gas 116, a screw 15 disposed in the cartridge 101 for moving a volume-displacing plunger 20a in the hollow barrel 7, a cap 67 with a closed distal end 68 and an elongate interior cavity 65 with a proximal open end 69 attached to the medical fluid delivery device 60a, a cartridge 101 including a hollow barrel 7 with a hollow proximal portion 30 with an interior wall 5 with a first inside diameter D1 with an open proximal end 30a and a funneled hollow distal end 25 with an funneled interior cavity 50 with an open distal end 41, a membrane 33 covering the open distal end 41 of the funneled hollow distal end 25, and a volume-displacing plunger 20a disposed inside the hollow barrel 7 through the open proximal end 30a of the hollow proximal portion 30 and configured to reduce the cubic volumetric capacity inside the hollow barrel 7 available for accommodating the a liquid or gas 116, the volume displacing plunger 20a including a solid proximal portion 39a with an exterior wall 29a with a first outside diameter equal to or exceeding the first inside diameter D1 of the interior wall 5 of the hollow proximal portion 30a and a funneled distal end 51 with a distal interior cavity 21 defined by a solid proximal interior wall 40 and an opposing opening 28 in a distal end wall 27, the solid proximal interior wall 40 configured between a least one axial interior side wall 52 and at least one opening 24 in at least one funneled exterior wall 70.

FIG. 2N illustrates a cross-sectional side view of the volume-displacing plunger 20e of the present invention comprising a distal end 51e with an interior cavity 21e, defined by a first opening 28e in a distal end wall 27f and an opposing solid proximal wall 40e, and a second opening 24e in the funneled exterior wall 70e and an opposing axial interior side wall 52h. The solid proximal wall 40e is configured between the axial interior side wall 52h and the conical exterior wall 35e of the conical portion 57e of the funneled exterior wall 70e. The axial interior side wall 52h is separated from the rigid exterior wall 10a, as shown in FIG. 2B, of the needle 10, at a minimum clearance distance of L4.

FIG. 2O is a full top view of the volume-displacing plunger 20a, of FIG. 2A, configured with a body 22 as shown in FIG. 2A, with a proximal portion 39a with a radial outside wall or radial ring 29a with an outside diameter ≥D1, and a distal end 51, configured with an funneled exterior wall 70 and a distal interior cavity 21, defined by a solid proximal interior wall 40 and an opposing opening 28, shown in a broken line, in a distal end wall 27, the solid proximal interior wall 40 is configured between a least one axial interior side wall 52 and at least one opening 24 as shown in FIG. 2P, in at least one funneled exterior wall 70. The second opening 24, is defined between the first axial interior wall 52 as shown in FIG. 2P, the second axial interior side wall 52a and the third opposing axial interior side wall 52b, and the solid proximal interior wall 40 and the opposing the first opening 28 configured in the distal end wall 27.

FIG. 2P is a full front view of the volume-displacing plunger 20a, of FIG. 2O, configured with a proximal portion 39a with at least one radial ring 29a, and a distal end 51 with an interior cavity 21, as shown in FIG. 2O, including a first opening 28, configured in a distal end wall 27, between a first axial interior wall 52 and at least one funneled exterior wall 70. The first opening 28 comprises a first axial interior wall 52, joined with a second axial interior side wall 52a and a third opposing axial interior side wall 52b. The solid proximal interior wall 40 is configured between a least one interior axial side wall 52 and at least one opening 24 in the funneled exterior wall 70.

FIG. 2Q is a full top view of the volume-displacing plunger 20c of FIGS. 2D and 2E, configured with a body 22c with a proximal portion 39b with a radial outside wall or ring 29a with an outside diameter ≥D1, as shown in FIG. 2O, and a funneled distal end 51c, configured with a funneled exterior wall 70c and a distal interior cavity 21c defined by sloped solid proximal wall 40c and an opposing opening 28c, shown as a broken line, in a distal end wall 27c. The body 22c comprises the solid proximal portion 39b, a conical portion 57c, configured with an exterior conical wall 35c, and an elongate distal portion 34c configured with axial exterior side wall 23c. The angled solid proximal interior wall 40c is configured between a least one axial interior side wall 52c, as shown in FIG. 2R, and at least one opening 24c in at least one funneled exterior wall 70c. The body 22c comprises a funneled distal end 51c configured by combining a conical portion 57c and an elongate distal portion 34c with a distal interior cavity 21c. The first opening 28c comprises a first non-radial axial interior wall 52c, as shown in FIG. 2R, joined with a second axial interior side wall 52d and a third opposing axial interior side wall 52e. The overall axial length of the volume-displacing plungers 20b, 20c, 20d, 20e and 220 of the present invention may be configured with an axial length of L7 as shown in FIG. 4L. The proximal portion 39b of the elastomeric volume-displacing plunger 20c comprises a medial radial ring 29d with an outside diameter D4, configured between substantially matching radial rings 29c with a reduced outside diameter D5, configured between substantially matching radial rings 29b and 29e with an outside diameter D3 exceeding the outside diameter D4 of the medial radial ring 29d.

FIG. 2R is a full front view of the volume-displacing plunger 20c, of FIG. 2Q, configured with a proximal portion 39b with at least one radial ring 29b. A distal end 51c with a distal interior cavity 21c is configured between the first opening 28c, in the distal end wall 27c and an opposing angled solid proximal wall 40c, and the second opening 24c configured in the funneled exterior wall 70c. The second opening 24c, is defined between the first axial interior wall 52c, the second axial interior side wall 52d and the third opposing axial interior side wall 52e as shown in FIG. 2Q, and the angled solid proximal wall 40c and the opposing the first opening 28c configured in the distal end wall 27c.

FIG. 2S is a full top view of the volume-displacing plunger 20d of FIG. 2F, comprising a body 22d with a proximal portion 39a with at least one radial ring 29a, and a funneled distal end 51d configured with a funneled exterior wall 70d and a distal interior cavity 21d. The funneled distal end 51d is configured with a distal interior cavity 21d comprising a first angled solid proximal interior wall 40d joined with a second angled solid proximal interior wall 40e as shown in FIG. 2T, and an opposing first opening 28d configured between a first distal end wall 27d and a second distal end wall 27e, a second opening 24d, configured in a first side of the funneled exterior wall 70d between a first axial interior side wall 52f and an opposing second axial interior side wall 52g, and an opposing third opening 24e, configured in the second opposing side of the funneled exterior wall 70d between the first axial interior side wall 52f and the opposing second axial interior side wall 52g. The body 22d comprises the solid proximal portion 39a, a conical portion 57d, configured with an exterior conical wall 35d, and an elongate distal portion 34d configured with axial exterior side wall 23d. The body 22d comprises the funneled distal end 51d configured by combining a conical portion 57d and an elongate distal portion with a distal interior cavity 21d.

FIG. 2T is a full front view of the volume-displacing plunger 20d, of FIG. 2S, configured with the funneled distal end 51d with a distal interior cavity 21d configured between a first solid proximal interior wall 40d joined with second solid proximal interior wall 40e and an opposing first opening 28d in a distal end wall 27d and, and a second opening 24d and a third opening 24d configured in between the first axial interior side wall 52f and the opposing second axial interior side wall 52g, as shown in FIG. 2S of the funneled distal end 51d.

FIGS. 3A and 3B illustrate the reduced-volume cartridge 201 of the present invention comprising a hollow barrel 7, available for accommodating liquid or gas 116, with unitarily formed volume-displacing plunger 120 moveable and translatable in the hollow barrel 7, having body 122 configured with combined interior through passages 121 and 121a, configured in a funneled distal end 151. The funneled distal end 151 comprises a funneled exterior side wall 170 and distal end wall 127 that may be configured to engage at least a portion of the funnel-shaped funneled interior wall 73 of the funneled interior cavity 50 of a funneled hollow distal end 25, forming first liquid-tight and air-tight seal 158 and a second liquid-tight and air-tight seal 147. The volume-displacing plunger 120 is configured to reduce the cubic volumetric measure of fluid or gas 116 within the hollow barrel 7 and funneled interior cavity 50 of the funneled hollow distal end 25, before, during and after the fluid or gas 116 is added to, or removed from the hollow barrel 7. The reduced-volume cartridge 201of the present invention may be configured with the specifications recommended by the ISO 13926-1:2018 standard.

According to one implementation of the present invention, FIG. 3A, illustrates a cross-sectional side view of the reduced-volume cartridge 201 of the present invention, configured with an elastomeric unitarily-formed volume-displacing plunger 120, disposed in a hollow barrel 7, reducing the cubic volumetric capacity available for accommodating liquid or gas 116. The reduced-volume cartridge 201 is configured with the hollow barrel 7 with a proximal portion 30, having an open proximal end 30a, joined with a distal portion 31, as shown in FIG. 1A, configured with a funnel-shaped funneled hollow distal end 25 with an open distal end 41. A membrane 33 covers the open distal end 41 of the funneled hollow distal end 25 of the hollow barrel 7. The unitarily-formed volume-displacing plunger 120 comprises a body 122, configured with a solid proximal portion 139, that may include at least one exterior protrusion or radial ring 129, with an outside diameter ≥D1, as shown in FIG. 3C. The at least one radial ring 129 is configured to form a least a first liquid-tight and air-tight seal 145 with the interior wall 5 of elongate tube 2, as shown in FIG. 3B. The body 122, comprises the solid proximal portion 139, a conical portion 157 and an elongate distal portion 134, as shown in FIG. 3C, and may be configured with a combined axial length of L7, as shown in FIG. 3C. The body 122, may comprise only a solid proximal portion 139 and an elongate portion 134. The interior conical wall 36, of the conical portion 37, as shown in FIG. 1A, and the short interior wall 6 of the narrow tube 13, as shown in FIG. 1A, of the hollow barrel 7, combine to form funneled interior wall 73, configured in the funneled hollow distal end 25.

The body 122, comprises a funneled distal end 151 configured with a conical portion 157, as shown in FIG. 3C, having an exterior conical wall 135, as shown in FIG. 3D, and an elongate distal portion 134, as shown in FIG. 3C, with an axial exterior side wall 123, as shown in FIG. 3D, and a distal end wall 127. The exterior conical wall 135 and the axial exterior wall 123, as shown in FIG. 3D, of the funneled distal end 151, are configured to form the funneled exterior wall 170, with a bend 171, as shown in FIG. 3D. The funneled distal end 151 comprises a first opening 124, of a first interior cavity 121, configured between a solid proximal interior wall 140 and an opposing solid interior wall 140a, as shown in FIG. 3B. The funneled distal end 151, includes a second through passage 121a, configured with a second opening 128, shown as a broken line, configured in a distal end wall 127, in communication with the first through passage 121. The solid proximal interior wall 140 intersects with the proximal end of the interior side wall 152, as shown in FIG. 3D, and the opposing solid interior wall 140a intersects with an interior side wall 152a as shown in FIG. 3D. The solid proximal interior wall 140 is configured between the proximal end of the interior side wall 152 and at least one opening 124 in at least one funneled exterior wall 170. The second opening 128 and the second through passage 121a are configured to receive a hollow needle 10, as shown in FIG. 2B. The first opening 124 does not intersect with the second opening 128. The membrane 33 may comprise a multi-compound elastomeric rubber liner configured to withstand multiple piercings and maintain a leak-proof profile. A second liquid-tight and air tight seal 42 is configured between a portion of the proximal wall 32, of the membrane 33, and the distal end wall 49 of funneled hollow distal end 25.

The first through passage 121 and the second through passage 121a may comprise an “L” configuration, configured between the first opening 124, in the funneled exterior wall 170, and the second opening 128 configured in the distal end wall 127. The through passages 121 and 121a, may also comprise an “T” or Y” configuration, configured between a first opening 124a, in the exterior side wall 170, second opening 128, in the distal end wall 127, and a third opening 124b, in an exterior side wall 170, opposing the first opening 124a. The first opening 124a, may be configured to extend into the exterior conical wall 135 wall of the exterior side wall 170, as shown in FIG. 2N.

A medical fluid cartridge 201 comprising a body 9 including a hollow barrel 7 with a hollow proximal portion 30 with an interior wall 5 with a first inside diameter D1 with an open proximal end 30a and a funneled hollow distal end 25 with a funneled interior cavity 50 with a distal open end 41, a membrane 33 covering the distal open end 41 of the funneled hollow distal end 25, and a unitarily-formed volume-displacing plunger 120 disposed inside the hollow barrel 7 through the open proximal end 30a of the hollow proximal portion 30 and configured to reduce the cubic volumetric capacity inside the hollow barrel 7 available for accommodating a liquid or gas 116, the unitarily-formed volume-displacing plunger 120 including a body 122 with a proximal portion 139 with an exterior wall 129 with a first outside diameter equal to or exceeding the first inside diameter D1 of the interior wall 5 of the hollow proximal portion 30 and a distal end 151 with a first through passage 121a configured with at least one opening 124 in at least one funneled exterior wall 170, the first through passage 121 in communication with a second through passage 121a configured with a second opening 128 in a distal end wall 127.

FIG. 3B illustrates a cross-sectional side view of the reduced-volume cartridge 201, of FIG. 3A, configured with a volume-displacing plunger 120 disposed in the funneled interior cavity 50 of the funneled hollow distal end 25 of the hollow barrel 7, as shown in FIG. 3A, available for accommodating a liquid or gas 116. The volume-displacing plunger 120 comprises a body 122, configured with a solid proximal portion 139, as shown in FIG. 3C, that may include at least one exterior protrusion or radial ring 129, with an outside diameter ≥D1, as shown in FIG. 3C. The at least one radial ring 129 is configured to engage the interior wall 5 of the elongate tube 2, forming a first liquid-tight and air-tight seal 145 with the interior wall 5 of elongate tube 2. As shown in FIG. 3A, a second liquid-tight and air tight seal 42 is configured between a portion of the proximal wall 32, of the membrane 33, and the distal end wall 49 of funneled hollow distal end 25. The funneled exterior wall 170, of the funneled distal end 151, is configured to engage the funneled interior wall 73 of the funneled hollow distal end 25, as shown in FIG. 3A, to form a third liquid-tight and air-tight seal 158 configured from the corner bend 14 of the body 9, as shown in FIG. 1A, to the distal end wall 49 of the funneled hollow distal end 25, as shown in FIG. 3A. As shown in FIG. 3A, the open end 41 of the funneled hollow distal end 25 is covered with a pierceable membrane 33. The distal end 151, comprises a distal end wall 127 configured to form a third liquid-tight and air tight seal 147 with at least a portion of a proximal facing wall 32 of membrane 33, as shown in FIG. 3A. The funneled distal end 151 comprises a first opening 124, of the interior cavity 121, configured between a solid proximal interior wall 140 and an opposing solid interior wall 140a. The funneled distal end 151, includes a second through passage 121a, configured with a second opening 128, configured in a distal end wall 12a, in communication with a first through passage 121.

A medical fluid dispensing apparatus 201 comprising a medical fluid delivery device 60b including a cartridge holder 17 holding a cartridge 301 with a hollow barrel 7 containing a liquid or gas 116; a pushrod 15 disposed in the cartridge 301 for moving a volume-displacing plunger 120 in the hollow barrel 7; a needle hub 18 with a needle 10 with a rigid exterior wall 10a with a sharpened proximal end 11a and sharpened distal end 11 with a hollow lumen 12 configured there between, the needle hub 18 including a hollow interior cavity 18a removably attached to a distal open end 63 of the cartridge holder 17, a cartridge 301 including a hollow barrel 7 with a hollow proximal portion 30 with an interior wall 5 with a first inside diameter D1 with an open proximal end 30a and a funneled hollow distal end 25 with a funneled interior cavity 50 with an open distal end 41, a membrane 33 covering the open distal end 41 of the funneled hollow distal end 25 and the sharpened proximal end 11a of the needle10 disposed in the funneled interior cavity 50 through the membrane 33, and a unitarily-formed volume-displacing plunger 120 disposed inside the hollow barrel 7 through the open proximal end 30a of the hollow proximal portion 30 and configured to reduce the cubic volumetric capacity inside the hollow barrel 7 available for accommodating the a liquid or gas 116, the unitarily-formed volume-displacing plunger 120 including a body 122 with a proximal portion 139 with an exterior wall 129 a first outside diameter equal to or exceeding the first inside diameter D1 of the interior wall 5 hollow proximal portion 30 and a distal end 151 having a first through passage 121 including at least one opening 124 in at least one funneled exterior wall 170, and a second through passage 121a including a first opening 128 in a distal end wall, the first through passage 121 in communication with the second through passage 121a.

FIG. 3C illustrates a partial cut-away view and partial cross-sectional side view of the unitarily-formed volume-displacing plunger 120, of FIG. 3A, configured with a body 122 with a proximal portion 139 with at least one exterior radial ring, 129, as shown in FIG. 3A, configured with an outside diameter ≥D1. The body 122, includes a funneled distal end 151, comprising a conical portion 157, with a conical exterior side wall 135, as shown in FIG. 3D, having an axial length of L3, as shown in FIG. 3D, combined with an elongate distal portion 134, configured an axial exterior side wall 123, as shown in FIG. 3D, with an outside diameter ≥D2 and length of L1. At least one radial ring may be configured about the periphery of the elongate distal portion 134, configured to form a liquid-tight and air-tight seal with the funneled interior wall 73, of the funneled hollow distal end 25, as shown in FIG. 3A. The overall axial length of the volume-displacing plungers 120 and 120a of the present invention may be configured with an axial length of L7.

FIG. 3D illustrates a cross-sectional side view of the unitarily-formed volume-displacing plunger 120, of FIG. 3A. comprising a funneled distal end 151, as shown in FIG. 3C. with a first through passage 121, as shown in FIG. 3C, configured with at least one opening 124 in a funneled exterior wall 170, as shown in FIG. 3A, and a second through passage 121a configured with a second opening 128 in a distal end wall 127 as shown in FIG. 3A. The first opening 124 and the first through passage 121, as shown in FIG. 3C, are configured between a solid proximal interior wall 140 and an opposing solid interior wall 140a. The solid proximal interior wall 140 intersects with the proximal end of the long axial interior side wall 152, and the opposing solid interior wall 140a intersects with the short axial interior side wall 152a. The distal end 151 comprises a funneled exterior wall 170, as shown in FIG. 3A, configured with a conical portion 157, as shown in FIG. 3C, and an elongate axial portion 134, as shown in FIG. 3C, with an axial exterior wall 123. A bend 171 is configured in the funneled exterior wall 170, as shown in FIG. 3A, at the intersection of the conical exterior wall 135 and the axial exterior side wall 123.

FIG. 3E illustrates a cross-sectional side view of the injection tool 80 to produce the unitarily-formed volume-displacing plunger 120 of FIG. 3A. The injection tool 80 comprises a first mold plate 81 with a first hollow gate 85a in communication with a first interior cavity 81a with a first narrow elongate cavity 92, and a second mold plate 82 configured with a second hollow gate 85b in communication with a second interior cavity 82a with a second narrow elongate cavity 93, machined by cutters and, if necessary, “burned” by electrical discharge machining electrodes forming the opposing exterior profiles of the volume displacing plunger 120 as shown in FIG. 3D. A removable solid core 83 is disposed in the narrow elongate portion 92 of the first mold plate 81 and the narrow elongate portion 93 of the second mold plate 82. The solid core 83 may include an exterior wall including a slight draft, tapering towards the shut off 84, and is configured to form a second opening 128, as shown FIG. 3A, and the interior profile of a second through passage 121a, as shown FIG. 3A, configured between a first interior side wall 152, as shown FIG. 3D, and a second opposing interior side wall 152a as shown FIG. 3D. The first mold plate 81 includes a solid block 88 configured to form a first opening 124, as shown FIG. 3A, and the interior profile of a first through passage 121, as shown in FIG. 3A, configured between the solid proximal interior wall 140 and the opposing solid interior wall 140a of the unitarily-formed volume-displacing plunger 120 as shown in FIG. 3D. When the solid block 88 engages the solid core 83, a shut-off 84 is configured at the intersection of the solid block 88 and the solid core 83, occupying the interior profile the interior cavity 121, as shown in FIG. 3D. When the first plate 81 and second plate 82 close together, the first interior cavity 81a communicatively opposes the second interior cavity 82a to fill the proximal portion 139, as shown in FIG. 3C, with molten plastic resin, and the first narrow elongate cavity 92 communicatively opposes the second narrow elongate cavity 93, filling the distal end 151 of the volume-displacing plunger 120, as shown in FIG. 3A, with molten plastic resin.

Ejector pins 91, in an ejector plate 90, are disposed through openings 89 configured in the first mold plate 81, with a length configured close off the exterior profile of the first interior cavity 81a and the exterior profile of the reduced elongate cavity 92. Springs 96 are disposed between the first mold plate 81 and the ejector plate 90. After the molten polymer resin has cured sufficiently to form the unitarily-formed volume-displacing plunger 120, as shown in FIG. 3C, the first mold plate 81 and second mold plate 82 separate and the core 83 is removed from the mold tool 80. An ejector retaining plate 95 pushes the ejector plate 90 and moves the ejector pins 91 into the first interior cavity 81a and the narrow elongate cavity 92 of the first mold plate 81, ejecting the solidified unitarily-formed volume-displacing plunger 120, as shown in FIG. 3C.

The method of monolithically forming a unitarily-formed volume-displacing plunger 120 including a proximal portion 139 and a distal end 151 with a first through passage 121 in communication with a second through passage 121a by filling molten plastic resin into an injection tool 80 including a first mold plate 81 with at least one hollow gate 85a for flowing molten plastic resin into a first interior cavity 81a with a first narrow elongate cavity 92 and the a second interior cavity 82a with a second narrow elongate cavity 93, the first mold plate 81 abutting a second mold plate 82 forming a parting line 86, the first interior cavity 81a communicatively opposing the second interior cavity 82a at the parting line 86 for filling the proximal portion 139 with molten plastic resin, and the first narrow elongate cavity 92 communicatively opposing the second narrow elongate cavity 93 at the parting line 86 for filling the distal end 151 with molten plastic resin, the first mold plate 81 having a solid block 88 disposed in a portion of the first narrow elongate cavity 92, the solid block extending into a portion of the second narrow elongate cavity 93 for forming a first through passage 121 in the distal end 151, and a removeable solid core 83 opposing the hollow gate 85a and disposed between the first narrow elongate cavity 92 communicatively opposing the second narrow elongate cavity 93 at the parting line 86 for forming a second through passage 121a in the distal end 151, the solid core 83 engaging the solid block 88 forming a shut-off 84, whereby the solid core 83 is removed from the injection tool 80 when the first mold plate 81 moves away from the second mold plate 82, and a retaining plate 95 abutting an ejector plate 90 with at least one ejector pin 91 disposed in and moving through at least one opening 89 in the first mold plate 81, the at least one moving ejector pin 91 ejecting the solidified unitarily-formed volume-displacing plunger 120 from the first interior cavity 81a and the first narrow elongate cavity 92 of the first mold plate 81, the unitarily-formed volume-displacing plunger 120 comprising a proximal portion 139 and a distal end 151 with a first through passage 121a in communication with a second through passage 121.

FIGS. 4A, 4D, 4E and 4K illustrate the reduced-volume cartridge 301 of the present invention comprising a hollow barrel 7, available for accommodating liquid or gas 116, with a volume-displacing plunger 220, moveable within the hollow barrel 7, having a body 222, with at least one elongate interior cavity 221 configured in a funneled distal end 251. The volume-displacing plunger 220 is configured to reduce the cubic volumetric measure of fluid or gas 116 within the hollow barrel 7, before, during or after the fluid or gas 116 is added to, or removed from the hollow barrel 7.

According to one implementation of the present invention, FIG. 4A illustrates a cross-sectional side view of the reduced-volume cartridge 301 of the present invention, configured with a volume-displacing plunger 220 displacing the majority of space in a funneled interior cavity 50, as shown in FIG. 1B, of the funneled hollow distal end 25 of the reduced volume-cartridge 301. The volume-displacing plunger 220 comprises a body 222, configured with a solid proximal portion 239, as shown in FIG. 4B, that may include at least one exterior protrusion or radial ring 229, with an outside diameter ≥D1, as shown in FIG. 4L. The at least one radial ring 229, is configured to engage the interior wall 5 of the elongate tube 2, forming a first liquid-tight and air-tight seal 245 with the interior wall 5 of elongate tube 2.

As shown in FIG. 1A, the reduced-volume cartridge 301 is configured with a hollow barrel 7 with a proximal portion 30, with an open proximal end 30a, joined with a distal portion 31 with a funneled hollow distal end 25 with an open distal end 41. The funneled interior cavity 50, of the funneled hollow distal end 25, is defined by a funneled interior wall 73, configured between the bend 14 of the body 9 and the distal end wall 49 of the narrow portion 13 as shown in FIG. 1A,

The volume-displacing plunger 220 is disposed in the hollow barrel 7, as shown in FIG. 1A, and is configured to reduce the space inside the hollow barrel 7 available for accommodating liquid or gas 116. The volume-displacing plunger 220 comprises a funneled distal end 251 configured with a conical portion 257 with an exterior conical wall 235 as shown in FIG. 4L, joined with an elongate distal portion 234, configured with an outside diameter ≥D2 as shown in FIG. 4L, defined by an axial exterior side wall 223, as shown in FIG. 4B. A funneled exterior wall 270 comprises the first exterior conical wall 235 as shown in FIG. 4B, joined with the second axial exterior side wall 223, as shown in FIG. 4B. The conical interior wall 235, as shown in FIG. 4B, and the short interior wall 6 of narrow tube 13 combine to form a funneled interior wall 73. The funneled distal end 251 comprises an elongate interior cavity 221, configured between a distal end wall 227 with a first opening 228 as shown in a broken line in FIG. 4B, and a second opening 224, as shown in a broken line in FIG. 4L, configured in the funneled exterior wall 270. The funneled exterior wall 270, of the funneled distal end 251 as shown in FIG. 4L, is configured to substantially engage the funneled interior wall 73 of the funneled interior cavity 50, as shown in FIG. 3A, of the funneled hollow distal end 25. When the funneled distal end 251 of volume-displacing plunger 220 is disposed in the funneled interior cavity 50 of the funneled hollow distal end 25, a second liquid-tight and air-tight seal 258 is configured from the corner bend 14, as shown in FIG. 1A, of the elongate tube 2, to the distal end wall 49 of the funneled hollow distal end 25. The axial exterior side wall 223, as shown in FIG. 4B, of the funneled exterior wall 270, of the funneled distal end 251, circumscribes the outside periphery of the funneled distal end 251 from first corner edge 253 to a second corner edge 254, as shown in FIG. 4C, forming a portion of the liquid-tight and air-tight seal 258, along the funneled interior wall 73 of the funneled hollow distal end 25 as shown in FIG. 3A. The open end 41 of the funneled hollow distal end 25, is covered with a pierceable membrane 33. The funneled distal end 251, of the body 222, comprises a distal end wall 227, may be configured to form a third liquid-tight and air tight seal 247 with at least a portion of a proximal facing wall 32 of membrane 33. A fourth liquid-tight and air tight seal 42, is configured between the proximal facing wall 32, of the membrane 33, and the distal end wall 49, of funneled hollow distal end 25 of the body 9, as shown in FIG. 1A.

FIG. 4B is a full top view of the volume-displacing plunger 220, of FIG. 4A, comprising a body 222, as shown in FIG. 4A, with a funneled distal end 251 configured with a proximal portion 239 with at least one radial outside wall or ring 229, and a conical portion 257, as shown in FIG. 4L, with a conical exterior wall 235. The funneled distal end 251 also comprises an elongate interior cavity 221, defined by a solid proximal interior wall 240 and an opposing first opening 228, shown in a broken line, in a distal end wall 227. The elongate interior cavity 221 and the first opening 228 are configured to receive a hollow needle 10, as shown in FIG. 4L, without the rigid exterior wall 10a of the needle, as shown in FIG. 4B, engaging the interior axial side wall 252, as shown in FIG. 4I, of the elongate interior cavity 221. The elongate interior cavity 221, includes a proximal interior end wall 240, sufficiently spaced from the sharpened proximal end 11a of the needle 10, as shown in FIG. 2B, without the sharpened proximal end 11a engaging the distal facing interior wall 240. The second opening 224 is configured in the funneled exterior wall 270 between a first axial interior wall 252a and an opposing axal interior wall 252b.

FIG. 4C is a full front view of the volume-displacing plunger 220, of FIG. 4B, comprising a body 222, as shown in FIG. 4A, with a proximal portion 239 with at least one exterior radial ring 229, as shown in FIG. 4B, and a funneled distal end 251 configured with an interior cavity 221, including a second opening 224, shown in a broken line, configured in the funneled exterior wall 270, configured between a first interior wall 252, a second axial interior side wall 252a and a third opposing axial interior side wall 252b, and at least one exterior side wall 270. The solid proximal interior wall 240, as shown in FIG. 4B, is configured between a least one axial interior side wall 252 and at least one opening 224 in the exterior side wall 270.

FIG. 4D illustrates a cross-sectional side view of the medical fluid dispensing apparatus 301A configured with the reduced-volume cartridge 301 disposed in the dispensing device 60c and the volume-displacing plunger 220 disposed in the hollow barrel 7, as shown in FIG. 1A, available for accommodating a liquid or gas 116. The reduced-volume cartridge 301 is disposed in the cartridge holder 17 of the dispensing device 60c joined with a needle hub 18 with a needle 10. The proximal end 11a of the needle 10, as shown in FIG. 2B, is disposed in the funneled interior cavity 50 of the funneled hollow distal end 25, as shown in FIG. 4A. The medical fluid dispensing device 60c is configured with a cartridge holder 17, and an open distal end 63 and a stop 64 as shown in FIG. 2B. The volume-displacing plunger 220, is disposed in the hollow barrel 7 as shown in FIG. 1A, and configured to reduce the space inside the hollow barrel 7 available for accommodating liquid or gas 116. When volume-displacing plunger 220 is moved proximally or distally in the hollow barrel 7, as shown in FIG. 1A, a flow path 238 is configured between the hollow barrel 7, in communication with the interior cavity 221, of the funneled distal end 251 as shown in FIG. 4B, in communication with the funneled interior cavity 50, of the funneled hollow distal end 25 as shown in FIG. 4A, and in communication with the lumen 12 of needle 10.

FIG. 4E illustrates a cross-sectional side view of the medical fluid dispensing apparatus 301B configured with the reduced-volume cartridge 301 disposed in the dispensing device 60c and the volume-displacing plunger 220 disposed in the funneled interior cavity 50 of the funneled hollow distal end 25 of the hollow barrel 7, as shown in FIG. 1A, available for accommodating a liquid or gas 116. A screw 15 of the dispensing device 60c has moved the funneled distal end 251 of the volume-displacing plunger 220 through the hollow barrel 7, as shown in FIG. 3A, and into the funneled interior cavity 50 of the funneled hollow distal end 25, as shown in FIG. 4A. The proximal end 11a of the needle 10, as shown in FIG. 2B, has pierced the membrane 33, as shown in FIG. 4A, and is disposed in the funneled interior cavity 50 of the funneled hollow distal end 25, as shown in FIG. 4A, and the interior cavity 221 of the funneled distal end 251. The funneled distal end 251b displaces the majority of the fluid 116, as shown in FIG. 4D, previously disposed in funneled interior cavity 50 of the funneled hollow distal end 25, as shown in FIG. 4A. of the funneled distal end 251. The interior cavity 221, of the funneled distal end 251, is now configured as a through passage 221, configured between the proximal facing wall 32, of the membrane 33 and the opposing distal facing interior wall 240 as shown in FIG. 4A, and the interior axial side wall 252, as shown in FIG. 4A, of the funneled distal end 251, and the short interior wall 6, as shown in FIG. 4A, of the opposing funneled interior wall 73 of the funneled hollow distal end 25 as shown in FIG. 4A. The funneled distal end 251 includes the interior through passage 221, in communication with the interior cavity 50, of the funneled hollow distal end 25, as shown in FIG. 4A, in communication with the needle lumen 12 of needle 10, as shown in FIG. 4B, forming flow path 238a.

FIG. 4F illustrates a cross-sectional front view of the dispensing apparatus 301A of FIG. 4D, in axis 4F-4F, with reduced-volume cartridge 301, as shown in FIG. 4D, disposed in the cartridge holder 17 of the dispensing device 60c, as shown in FIG. 4D. The fluid 116 is disposed in the funneled interior cavity 50, defined by the funneled interior wall 73, of the funneled hollow distal end 25, of the body 9, as shown in FIG. 1A, of the reduced-volume cartridge 301 of FIG. 4D.

FIG. 4G illustrates a cross-sectional front view of the medical fluid dispensing apparatus 301A of FIG. 4D, in axis 4G-4G, with the reduced-volume cartridge 301 disposed within the cartridge holder 17, as show in FIG. 4D. The fluid 116 is disposed in the narrow tube portion 13, as shown in FIG. 4A, of the funneled interior cavity 50, defined by the funneled interior wall 73, of the funneled hollow distal end 25 of the body 9, as shown in FIG. 1A. The proximal end 11a of needle 10 of needle hub 18, as shown in FIG. 1C, is disposed in the funneled interior cavity 50, in communication with the lumen 12, of the needle 10, as shown in FIG. 4D. A cap 4 secures the membrane 33, as shown in FIG. 4A, over the open end 41 of the funneled hollow distal end 25, as shown in FIG. 4A.

FIG. 4H illustrates a cross-sectional front view of the medical fluid dispensing apparatus 301B of FIG. 4E, in axis 4H-4H, with the reduced-volume cartridge 301 disposed within the cartridge holder 17, as show in FIG. 4D. The conical portion 257, as shown in FIG. 4L, of the funneled distal end 251 of the volume-displacing plunger 220, displaces the entirety of space within the conical interior cavity portion 48, as shown in FIG. 1A, of the funneled interior cavity 50 of the funneled hollow distal end 25, as shown in FIG. 4A. A liquid-tight and air-tight seal 258 is configured between the funneled exterior wall 270 of the funneled distal end 251, and the funneled interior wall 73 of the funneled hollow distal end 25 as shown in FIG. 4A.

FIG. 4I illustrates a cross-sectional front view of the medical fluid dispensing apparatus 301B of FIG. 4E, in axis 4I-4I, with the reduced-volume cartridge 301, as shown in FIG. 4E disposed in the cartridge holder 17. The funneled distal end 251 of the volume-displacing plunger 220, as shown in FIG. 4E, is disposed in the funneled interior cavity 50 of the funneled hollow distal end 25 as show in FIG. 4A. The funneled distal end 251, displaces the majority of fluid 116, as shown in FIG. 4D, previously disposed in the funneled interior cavity 50 of the funneled hollow distal end 25 as shown in FIG. 4A. The proximal end 11a of needle 10 of needle hub 18, as shown in FIG. 1C, is disposed in the funneled interior cavity 50, in communication with the interior through passage 221, as shown in FIG. 4E, of the funneled distal end 251, in communication with the needle lumen 12 as shown in FIG. 4E. A cap 4 secures a membrane 33 over the open end 41 of the funneled hollow distal end 25 as shown in FIG. 1A. A liquid-tight and air-tight seal 258 circumscribes the outside periphery of the funneled distal end 251, from the first corner edge 253 to the second corner edge 254 of the funneled distal end 251, between the narrow tube 13 portion of the funneled interior wall 73, of the funneled hollow distal end 25 as shown in FIG. 4A, and the exterior side wall 270 of the funneled distal end 251. The axial interior side wall 252, of the interior cavity/through passage 221, as shown in FIG. 4E, is spaced from the outside wall 10a, as shown in FIG. 2B of the needle 10, at a distance of L4, as shown in FIG. 2N.

FIG. 4J illustrates an isometric view of one implementation of the volume-displacing plunger 220 of the present invention comprises a body 222, as shown in FIG. 4A, configured with a solid proximal portion 239, that may include at least one protrusion of radial ring 229, as shown in FIG. 4A, and a funneled distal end 251 comprising a conical portion 257 as shown in FIG. 4L, with an exterior conical wall 235, as shown in FIG. 4B, and an elongate distal portion 234, as shown in FIG. 4L, with an axial exterior side wall 223, as shown in 4B. The axial exterior side wall 223, as shown in FIG. 4B, and the exterior conical wall 235, as shown in FIG. 4B combine to form the exterior side wall 270 of the funneled distal end 251, as shown in FIG. 4L. The distal end wall 227, as shown in FIG. 4B, is configured with a first opening 228, intersecting with at least one second opening 224, configured in the elongate distal portion 234, as shown in FIG. 4L, of the funneled exterior wall 270 of the funneled distal end 251, as shown in FIG. 4L. The funneled exterior wall 270 is configured with an intermediate bend 271 configured to substantially mirror the in the intermediate bend 43, as show in FIG. 1A, in the funneled interior wall 73 of the funneled interior cavity 50 of the funneled hollow distal end 25 as shown in FIG. 4A. The funneled distal end 251 comprises an interior cavity 221, as shown in FIG. 4L, defined by a solid proximal interior wall 240 and an opposing opening 228 in a distal end wall 227, as shown in FIG. 4B, the solid proximal interior wall 240 configured between a least one axial interior side wall 252, as shown in FIG. 4I, and at least one opening 224 in at least one exterior side wall 270.

FIG. 4K illustrates a cross-sectional side view of the medical fluid delivery apparatus 301C of the present invention. The medical fluid delivery apparatus 301C comprising a medical fluid delivery device 60b including a cartridge holder 17a with a stop 17b holding a lip 61 of a screw cap 44 attached to a cartridge 301 with a hollow barrel 7 containing a liquid or gas 116, a screw 15 disposed in the cartridge 1 for moving a volume-displacing plunger 220 in the hollow barrel 7, a cap 67 with a closed distal end 68 and an elongate interior cavity 65 with a proximal open end 69 attached to the medical fluid delivery device 60b, a cartridge 301 including a hollow barrel 7 with a hollow proximal portion 30 with an interior wall 5 with a first inside diameter D1 with an open proximal end 30a and a funneled hollow distal end 25 with a funneled interior cavity 50 with an open distal end 41, a membrane 33 covering the open distal end 41 of the funneled hollow distal end 25, and a volume-displacing plunger 220 disposed inside the hollow barrel 7 through the open proximal end 30a of the hollow proximal portion 30 and configured to reduce the cubic volumetric capacity inside the hollow barrel 7 available for accommodating the a liquid or gas 116, the volume-displacing plunger 220 including a body 222 with a solid proximal portion 239 with at least one exterior radial ring 229 with a first outside diameter equal to or exceeding the first inside diameter D1 of the interior wall 5 of the hollow proximal portion 30a and a distal end 251 with a distal interior cavity 221 defined by a solid proximal interior wall 240 and an opposing opening 228 in a distal end wall 227, the solid proximal interior wall 240 configured between a least one axial interior side wall 252 and at least one opening 224 in at least one funneled exterior wall 270.

FIG. 4L illustrates a cross-sectional side view volume-displacing plunger 220 of the present invention comprising a body 222, as shown in FIG. 4A, configured with a solid proximal portion 239, a conical portion 257 and an elongate distal portion 234, with an overall axial length of L7. The solid proximal portion 239, may include at least one radial exterior ring 229, as shown in FIG. 4B, and is configured with an outside diameter ≥D1. The conical exterior wall 235 of the exterior side wall 270, as shown in FIG. 4B, is configured at an obtuse angle, relative to the axis of the needle 10. The elongate distal portion 234 includes an exterior side wall 223, as shown in FIG. 4B, configured with an outside diameter ≥D2 and length of at least L1, as shown in FIG. 3B. The interior cavity 221 is defined by a first opening 228 configured in the distal end wall 227 and an opposing proximal interior wall 240 as shown in FIG. 4B, and by a second opening 224 configured in an exterior side wall 270 as shown in FIG. 4B, and an opposing axial interior wall 252 as shown in FIG. 4C. The axial interior side wall 252, as shown in FIG. 4I, of the elongate interior cavity 221, is sufficiently spaced from the rigid exterior wall 10a, as shown in FIG. 1B, of the needle 10, without the rigid exterior wall 10a engaging the axial interior side wall 252, as shown in FIG. 4I.

Numerous exemplary implementations have been disclosed and described herein. It is to be appreciated however, that the present invention is in no way to be construed as to being limited to these examples.

1. A cartridge for accommodating medical fluids comprising:
a body including a hollow barrel with a hollow proximal portion with an interior wall with a first inside diameter with an open proximal end and a funneled hollow distal end with a funneled interior cavity with a distal open end;
a membrane covering the distal open end of the funneled hollow distal end; and
a volume-displacing plunger disposed inside the hollow barrel through the open proximal end and configured to reduce the cubic volumetric capacity inside the hollow barrel available for accommodating a liquid or gas, the volume displacing plunger including a body with a solid proximal portion with an exterior wall a first outside diameter equal to or exceeding the first inside diameter of the interior wall of the hollow proximal portion and a funneled distal end having a distal interior cavity defined by a solid proximal interior wall and an opposing opening in a distal end wall, the solid proximal interior wall configured between a least one axial interior side wall and at least one opening in at least one funneled exterior wall.

2. The cartridge according to claim 1, wherein the solid proximal portion of the body includes at least one radial ring adapted to form a first liquid-tight and air-tight seal with the interior wall of the proximal portion of the hollow barrel.

3. The cartridge according to claim 2, wherein a proximal facing end of the membrane is adapted to close the distal open end of the funneled interior cavity of the funneled hollow distal end and form a second liquid-tight and air-tight seal with the distal end wall of the funneled hollow distal end.

4. The cartridge according to claim 3, wherein a cap with an open end is crimped over a lip of the funneled hollow distal end to secure the membrane over the distal open end of the funneled interior cavity of the funneled hollow distal end.

5. The cartridge according to claim 1, wherein the funneled hollow distal end includes a narrow tube with an inside wall with a second smaller inside diameter.
